(19) United States
(12) Patent Application Publication
(10) Pub. No.: US 2011/0104055 A1
(43) Pub. Date: May 5, 2011

(54) SUBSTITUTED N-ARYL BENZAMIDES AND RELATED COMPOUNDS FOR TREATMENT OF AMYLOID DISEASES AND SYNUCLEINOPATHIES

(76) Inventors: Alan Snow, Lynnwood, WA (US); Manfred Weigele, Cambridge, MA (US); Lesley Larsen, Dunedin (NZ); Beth Nguyen, Gurnee, IL (US); Thomas Lake, Snohomish, WA (US); Gerardo Castillo, Bothell, WA (US); Virginia Sanders, San Francisco, CA (US); Rex T. Weavers, Dunedin (NZ); Stephen Lorimer, Dunedin (NZ); David Larsen, Dunedin (NZ); David L. Coffen, (US); Charlotte Coffen, Belcamp, MD (US)

(21) Appl. No.: 12/940,330

(22) Filed: Nov. 5, 2010

Related U.S. Application Data
(63) Continuation-in-part of application No. 12/201,183, filed on Aug. 29, 2008, now abandoned.
Application No. 12/201,183 is a continuation-in-part of application No. 11/328,748, filed on Jan. 9, 2006, now Pat. No. 7,745,490.
Application No. 11/328,748 is a continuation of application No. 11/129,771, filed on May 12, 2005, now abandoned.
Application No. 11/129,771 is a continuation-in-part of application No. 12/837,721, filed on Jul. 16, 2010.
Application No. 12/837,721 is a continuation of application No. 12/269,017, filed on Nov. 11, 2008, now abandoned.
Application No. 12/269,017 is a continuation of application No. 10/452,851, filed on May 30, 2003, now Pat. No. 7,514,583.
(60) Provisional application No. 60/570,669, filed on May 12, 2004.
(60) Provisional application No. 60/629,525, filed on Nov. 18, 2004.
(60) Provisional application No. 60/463,104, filed on Apr. 14, 2003.
(60) Provisional application No. 60/435,880, filed on Dec. 20, 2002.
(60) Provisional application No. 60/412,272, filed on Sep. 20, 2002.
(60) Provisional application No. 60/409,100, filed on Sep. 9, 2002.
(60) Provisional application No. 60/385,144, filed on May 31, 2002.

(51) Int. Cl. A61K 31/167; C07C 233/75; A61K 51/00; A61P 43/00

(52) U.S. Cl. 424/1.65; 564/179; 514/622

(57) ABSTRACT

Compounds and their pharmaceutically acceptable salts, their synthesis and labeling, pharmaceutical compositions containing them, and their use in the treatment of amyloid diseases, including Aβ amyloidosis, such as observed in Alzheimer's disease, IAPP amyloidosis, such as observed in type 2 diabetes, and synucleinopathies, such as observed in Parkinson's disease, and the manufacture of medicaments for such treatment are provided. Also provided is the use of the disclosed compounds as imaging agents and methods for in vivo imaging and detection of amyloid and synuclein.

### RELATED APPLICATIONS

This application is a continuation in part of U.S. application Ser. No. 12/201,183 filed Aug. 29, 2008 which is a continuation in part of U.S. application Ser. No. 11/328,748, now issued U.S. Pat. No. 7,745,490, which is a continuation of U.S. application Ser. No. 11/129,771, filed May 12, 2005, entitled “Substituted N-Aryl Benzamides and Related Compounds for Treatment of Amyloid Diseases and Synucleinopathies” now abandoned. U.S. application Ser. No. 11/129,771 claimed priority under 35 U.S.C. §119(e) to U.S. provisional application Ser. Nos. 60/570,669, entitled “Substituted N-Aryl Benzamides and Related Compounds for Treatment of Amyloid Diseases and Synucleinopathies” to Snow et al., filed May 12, 2004 and 60/629,525, entitled “Substituted N-Aryl Benzamides and Related Compounds for Treatment of Amyloid Diseases and Synucleinopathies” to Snow et al., filed Nov. 18, 2004.

This application is also a continuation in part of U.S. application Ser. No. 12/837,721 filed Jul. 16, 2010 which is a continuation of U.S. application Ser. No. 12/269,017 filed Nov. 11, 2008 which is a continuation of, U.S. application Ser. No. 10/452,851, filed May 30, 2003, now issued U.S. Pat. No. 7,514,583 which claims priority under 35 USC 119(e) to:
(1) U.S. Provisional Application No. 60/385,144, filed May 31, 2002,
(2) U.S. Provisional Application No. 60/409,100, filed Sep. 9, 2002,
(3) U.S. Provisional Application No. 60/412,272, filed Sep. 20, 2002,
(4) U.S. Provisional Application No. 60/435,880, filed Dec. 20, 2002, and
(5) U.S. Provisional Application No. 60/463,104, filed Apr. 14, 2003.

The contents of the above-referenced applications are incorporated by reference herein.

### TECHNICAL FIELD

Provided herein are substituted N-aryl benzamides and related compounds, pharmaceutical compositions and methods for treatment of amyloid diseases, including beta-amyloid protein (Aβ), such as observed in Alzheimer's disease and Down's syndrome, islet amyloid polypeptide (IAPP), such as observed in type 2 diabetes, and alpha-synuclein, such as observed in Parkinson's disease.

### BACKGROUND

Alzheimer's disease is characterized by the accumulation of a 39-43 amino acid peptide termed the β-amyloid protein or Aβ, in a fibrillar form, existing as extracellular amyloid plaques and as amyloid within the walls of cerebral blood vessels. Fibrillar Aβ amyloid deposition in Alzheimer's disease is believed to be detrimental to the patient and eventually leads to toxicity and neuronal cell death, characteristic hallmarks of Alzheimer's disease. Accumulating evidence implicates amyloid, and more specifically, the formation, deposition, accumulation and/or persistence of Aβ fibrils, as a major causative factor of Alzheimer's disease pathogenesis. In addition, besides Alzheimer's disease, a number of other amyloid diseases involve formation, deposition, accumulation and persistence of Aβ fibrils, including Down's syndrome, disorders involving congophilic angiopathy, such as but not limited to, hereditary cerebral hemorrhage of the Dutch type, inclusion body myositosis, dementia pugilistica, cerebral β-amyloid angiopathy, dementia associated with progressive supranuclear palsy, dementia associated with cortical basal degeneration and mild cognitive impairment.

Parkinson's disease is another human disorder characterized by the formation, deposition, accumulation and/or persistence of abnormal fibrillar protein deposits that demonstrate many of the characteristics of amyloid. In Parkinson's disease, an accumulation of cytoplasmic Lewy bodies consisting of filaments of α-synuclein/NAC (non-Aβ component) are believed important in the pathogenesis and as therapeutic targets. New agents or compounds able to inhibit α-synuclein and/or NAC formation, deposition, accumulation and/or persistence, or disrupt pre-formed α-synuclein/NAC fibrils (or portions thereof) are regarded as potential therapeutics for the treatment of Parkinson's and related synucleinopathies. NAC is a 35 amino acid fragment of α-synuclein that has the ability to form amyloid-like fibrils either in vitro or as observed in the brains of patients with Parkinson's disease. The NAC fragment of α-synuclein is a relative important therapeutic target as this portion of α-synuclein is believed crucial for formation of Lewy bodies as observed in all patients with Parkinson's disease, synucleinopathies and related disorders.

A variety of other human diseases also demonstrate amyloid deposition and usually involve systemic organs (i.e. organs or tissues lying outside the central nervous system), with the amyloid accumulation leading to organ dysfunction or failure. These amyloid diseases (discussed below) leading to marked amyloid accumulation in a number of different organs and tissues, are known as systemic amyloidoses. In other amyloid diseases, single organs may be affected such as the pancreas in 90% of patients with type 2 diabetes. In this type of amyloid disease, the beta-cells in the islets of Langerhans in pancreas are believed to be destroyed by the accumulation of fibrillar amyloid deposits consisting primarily of a protein known as islet amyloid polypeptide (IAPP). Inhibiting or reducing such IAPP amyloid fibril formation, deposition, accumulation and persistence is believed to lead to new effective treatments for type 2 diabetes. In Alzheimer's disease, Parkinson's and “systemic” amyloid diseases, there is currently no cure or effective treatment, and the patient usually dies within 3 to 10 years from disease onset.

The amyloid diseases (amyloidoses) are classified according to the type of amyloid protein present as well as the underlying disease. Amyloid diseases have a number of common characteristics including each amyloid consisting of a unique type of amyloid protein. The amyloid diseases include, but are not limited to, the amyloid associated with Alzheimer's disease, Down's syndrome, hereditary cerebral hemorrhage with amyloidosis of the Dutch type, dementia pugilistica, inclusion body myositosis (Askanas et al, Ann. Neurol. 43:521-560, 1993) and mild cognitive impairment (where the specific amyloid is referred to as beta-amyloid protein or Aβ), the amyloid associated with chronic inflammation, various forms of malignancy and Familial Mediterranean Fever (where the specific amyloid is referred to as AA amyloid or inflammation-associated amyloidosis), the amyloid associated with multiple myeloma and other B-cell dyscrasias (where the specific amyloid is referred to as AL amyloid), the amyloid associated with type 2 diabetes (where the specific amyloid protein is referred to as amylin or islet amyloid polypeptide or IAPP), the amyloid associated with the prion diseases including Creutzfeldt-Jakob disease, Gerstmann-Straussler syndrome, kuru and animal scrapie (where the specific amyloid is referred to as PrP amyloid), the amyloid associated with long-term hemodialysis and carpal tunnel syndrome (where the specific amyloid is referred to as α₂-microglobulin amyloid), the amyloid associated with senile cardiac amyloidosis and Familial Amyloidotic Polyneuropathy (where the specific amyloid is referred to as transthyretin or prealbumin), and the amyloid associated with endocrine tumors such as medullary carcinoma of the thyroid (where the specific amyloid is referred to as variants of procalcitonin). In addition, the α-synuclein protein which forms amyloid-like fibrils, and is Congo red and Thioflavin S positive (specific stains used to detect amyloid fibrillar deposits), is found as part of Lewy bodies in the brains of patients with Parkinson's disease, Lewy body disease (Lewy in Handbuch der Neurologie, M. Lewandowski, ed., Springer, Berlin pp. 920-933, 1912; Pollanen et al, J. Neuropath. Exp. Neurol. 52:183-191, 1993; Spillantini et al, Proc. Natl. Acad. Sci. USA 95:6469-6473, 1998; Arai et al, Neurosci. Lett. 259:83-86, 1999), multiple system atrophy (Wakabayashi et al, Acta Neuropath. 96:445-452, 1998), dementia with Lewy bodies, and the Lewy body variant of Alzheimer's disease. For purposes of this disclosure, Parkinson's disease, due to the fact that fibrils develop in the brains of patients with this disease (which are Congo red and Thioflavin S positive, and which contain predominant beta-pleated sheet secondary structure), is now regarded as a disease that also displays the characteristics of an amyloid-like disease.

Systemic amyloidoses which include the amyloid associated with chronic inflammation, various forms of malignancy and familial Mediterranean fever (i.e. AA amyloid or inflammation-associated amyloidosis) (Benson and Cohen, Arth. Rheum. 22:36-42, 1979; Kamei et al, Acta Path. Jpn. 32:123-133, 1982; McAdam et al., Lancet 2:572-573, 1975; Metaxas, Kidney Int 20:676-685, 1981), and the amyloid associated with multiple myeloma and other B-cell dyscrasias (i.e. AL amyloid) (Harada et al., J. Histochem. Cytochem. 19:1-15, 1971), as examples, are known to involve amyloid deposition in a variety of different organs and tissues generally lying outside the central nervous system. Amyloid deposition in these diseases may occur, for example, in liver, heart, spleen, gastrointestinal tract, kidney, skin, and/or lungs (Johnson et al, N. Engl. J. Med. 321:513-518, 1989). For most of these amyloidoses, there is no apparent cure or effective treatment and the consequences of amyloid deposition can be detrimental to the patient. For example, amyloid deposition in the kidney may lead to renal failure, whereas amyloid deposition in the heart may lead to heart failure. For these patients, amyloid accumulation in systemic organs leads to eventual death generally within 3-5 years. Other amyloidoses may affect a single organ or tissue such as observed with the Aβ amyloid deposits found in the brains of patients with Alzheimer's disease and Down's syndrome: the PrP amyloid deposits found in the brains of patients with Creutzfeldt-Jakob disease, Gerstmann-Straussler syndrome, and kuru; the islet amyloid (IAPP) deposits found in the islets of Langerhans in the pancreas of 90% of patients with type 2 diabetes (Johnson et al, N. Engl. J. Med. 321:513-518, 1989; Lab. Invest. 66:522 535, 1992); the α₂-microglobulin amyloid deposits in the medial nerve leading to carpal tunnel syndrome as observed in patients undergoing long-term hemodialysis (Geyjo et al, Biochem. Biophys. Res. Comm. 129:701-706, 1985; Kidney Int. 30:385-390, 1986); the prealbumin/transthyretin amyloid observed in the hearts of patients with senile cardiac amyloid; and the prealbumin/transthyretin amyloid observed in peripheral nerves of patients who have familial amyloidotic polyneuropathy (Skinner and Cohen, Biochem. Biophys. Res. Comm. 99:1326-1332, 1981; Saraiva et al, J. Lab. Clin. Med. 102:590-603, 1983; J. Clin. Invest. 74:104-119, 1984; Tawara et al, J. Lab. Clin. Med. 98:811-822, 1989).

Alzheimer's disease also puts a heavy economic burden on society. A recent study estimated that the cost of caring for one Alzheimer's disease patient with severe cognitive impairments at home or in a nursing home, is more than $47,000 per year (A Guide to Understanding Alzheimer's Disease and Related Disorders). For a disease that can span from 2 to 20 years, the overall cost of Alzheimer's disease to families and to society is staggering. The annual economic toll of Alzheimer's disease in the United States in terms of health care expenses and lost wages of both patients and their caregivers is estimated at $80 to $100 billion (2003 Progress Report on Alzheimer's Disease).

Amyloid as a Therapeutic Target for Alzheimer's Disease

Alzheimer's disease is characterized by the deposition and accumulation of a 39-43 amino acid peptide termed the beta-amyloid protein, Aβ or β/A4 (Glenner and Wong, Biochem. Biophys. Res. Comm. 120:885-890, 1984; Masters et al., Proc. Natl. Acad. Sci. USA 82:4245-4249, 1985; Husby et al., Bull. WHO 71:105-108, 1993). Aβ is derived by protease cleavage from larger precursor proteins termed β-amyloid precursor proteins (APPs) of which there are several alternatively spliced variants. The most abundant forms of the APPs include proteins consisting of 695, 751 and 770 amino acids (Tanzi et al., Nature 31:528-530, 1988).

The small Aβ peptide is a major component that makes up the amyloid deposits of “plaques” in the brains of patients with Alzheimer's disease. In addition, Alzheimer's disease is characterized by the presence of numerous neurofibrillary “tangles”, consisting of paired helical filaments which abnormally accumulate in the neuronal cytoplasm (Grundke-Iqbal et al., Proc. Natl. Acad. Sci. USA 83:4913-4917, 1986; Kosik et al., Proc. Natl. Acad. Sci. USA 83:4044-4048, 1986; Lee et al., Science 251:675-678, 1991). The pathological hallmark of Alzheimer's disease is therefore the presence of “plaques” and “tangles”, with amyloid being deposited in the central core of the plaques. The other major type of lesion found in the Alzheimer's disease brain is the accumulation of amyloid in the walls of blood vessels, both within the brain parenchyma and in the walls of meningeal vessels that lie outside the brain. The amyloid deposits localized to the walls of blood vessels are referred to as cerebrovascular amyloid or congophilic angiopathy (Mandybur, J. Neuropath. Exp. Neurol. 45:79-90, 1986; Pardridge et al., J. Neurochem. 49:1394-1401, 1987)

For many years there has been an ongoing scientific debate as to the importance of “amyloid” in Alzheimer's disease, and whether the “plaques” and “tangles” characteristic of this disease were a cause or merely a consequence of the disease. Within the last few years, studies now indicate that amyloid is indeed a causative factor for Alzheimer's disease and should not be regarded as merely an innocent bystander. The Alzheimer's Aβ protein in cell culture has been shown to cause degeneration of nerve cells within short periods of time (Pike et al., Br. Res. 563:311-314, 1991; J. Neurochem. 64:253-265, 1995). Studies suggest that it is the fibrillar structure (consisting of a predominant β-pleated sheet secondary structure), characteristic of all amyloids, that is responsible for the neurotoxic effects. Aβ has also been found to be neurotoxic in slice cultures of hippocampus (Harrigan et al., Neurobiol. Aging 16:779-789, 1995) and induces nerve cell death in transgenic mice (Games et al., Nature 373:523-527, 1995; Hsiao et al., Science 274:99-102, 1996). Injection of the Alzheimer's Aβ into rat brain also causes memory impairment and neuronal dysfunction (Flood et al., Proc. Natl. Acad. Sci. USA 88:3363-3366, 1991; Br. Res. 663:271-276, 1994).

Probably, the most convincing evidence that Aβ amyloid is directly involved in the pathogenesis of Alzheimer's disease comes from genetic studies. It was discovered that the production of Aβ can result from mutations in the gene encoding, its precursor, β-amyloid precursor protein (Van Broeckhoven et al., Science 248:1120-1122, 1990; Murrell et al., Science 254:97-99, 1991; Haass et al., Nature Med. 1:1291-1296, 1995). The identification of mutations in the beta-amyloid precursor protein gene that cause early onset familial Alzheimer's disease is the strongest argument that amyloid is central to the pathogenetic process underlying this disease. Four reported disease-causing mutations have been discovered which demonstrate the importance of Aβ in causing familial Alzheimer's disease (reviewed in Hardy, Nature Genet. 1:233-234, 1992). All of these studies suggest that providing a drug to reduce, eliminate or prevent fibrillar Aβ formation, deposition, accumulation and/or persistence in the brains of human patients will serve as an effective therapeutic.

Parkinson's Disease and Synucleinopathies

Parkinson's disease is a neurodegenerative disorder that is pathologically characterized by the presence of intracytoplasmic Lewy bodies (Lewy in Handbuch der Neurologie, M. Lewandowski, ed., Springer, Berlin, pp. 920-933, 1912; Pollanen et al., J. Neuropath. Exp. Neurol. 52:183-191, 1993), the major components of which are filaments consisting of α-synuclein (Spillantini et al., Proc. Natl. Acad. Sci. USA 95:6469-6473, 1998; Arai et al., Neurosci. Lett. 259:83-86, 1999), an 140-amino acid protein (Ueda et al., Proc. Natl. Acad. Sci. USA 90:11282-11286, 1993). Two dominant mutations in α-synuclein causing familial early onset Parkinson's disease have been described suggesting that Lewy bodies contribute mechanistically to the degeneration of neurons in Parkinson's disease and related disorders (Polymeropoulos et al., Science 276:2045-2047, 1997; Kruger et al., Nature Genet. 18:106-108, 1998). Recently, in vitro studies have demonstrated that recombinant α-synuclein can indeed form Lewy body-like fibrils (Conway et al., Nature Med. 4:1318-1320, 1998; Hashimoto et al., Brain Res. 799:301-306, 1998; Nahri et al., J. Biol. Chem. 274:9843-9846, 1999). Most importantly, both Parkinson's disease-linked α-synuclein mutations accelerate this aggregation process, demonstrating that such in vitro studies may have relevance for Parkinson's disease pathogenesis. Alpha-synuclein aggregation and fibril formation fulfills of the criteria of a nucleation-dependent polymerization process (Wood et al., J. Biol. Chem. 274:19509-19512, 1999). In this regard α-synuclein fibril formation resembles that of Alzheimer's β-amyloid protein (Aβ) fibrils. Alpha-synuclein recombinant protein, and non-Aβ component (known as NAC), which is a 35-amino acid peptide fragment of α-synuclein, both have the ability to form fibrils when incubated at 37° C., and are positive with amyloid stains such as Congo red (demonstrating a red/green birefringence when viewed under polarized light) and Thioflavin S (demonstrating positive fluorescence) (Hashimoto et al., Brain Res. 799:301-306, 1998; Ueda et al., Proc. Natl. Acad. Sci. USA 90:11282-11286, 1993).

Synucleins are a family of small, presynaptic neuronal proteins composed of α-, β-, and γ-synucleins, of which only α-synuclein aggregates have been associated with several neurological diseases (Ian et al., Clinical Neurosc. Res. 1:445-455, 2001; Trojanowski and Lee, Neurotoxicology 23:457-460, 2002). The role of synucleins (and in particular, alpha-synuclein) in the etiology of a number of neurodegenerative and/or amyloid diseases has developed from several observations. Pathologically, synuclein was identified as a major component of Lewy bodies, the hallmark inclusions of Parkinson's disease, and a fragment thereof was isolated from amyloid plaques of a different neurological disease, Alzheimer's disease. Biochemically, recombinant α-synuclein was shown to form amyloid-like fibrils that recapitulated the ultrastructural features of alpha-synuclein isolated from patients with dementia with Lewy bodies, Parkinson's disease and multiple system atrophy. Additionally, the identification of mutations within the synuclein gene, albeit in rare cases of familial Parkinson's disease, demonstrated an unequivocal link between synuclein pathology and neurodegenerative diseases. The common involvement of α-synuclein in a spectrum of diseases such as Parkinson's disease, dementia with Lewy bodies, multiple system atrophy and the Lewy body variant of Alzheimer's disease has led to the classification of these diseases under the umbrella term of “synucleinopathies”.

Parkinson's disease α-synuclein fibrils, like the Aβ fibrils of Alzheimer's disease, also consist of a predominantly β-pleated sheet structure. Therefore, compounds found to inhibit Alzheimer's disease Aβ amyloid fibril formation are also anticipated to be effective in the inhibition of α-synuclein/NAC fibril formation, as shown from Examples provided herein. These compounds would therefore also serve as therapeutics for Parkinson's disease and other synucleinopathies, in addition to having efficacy as a therapeutic for Alzheimer's disease, type 2 diabetes, and other amyloid disorders.

Discovery and identification of new compounds or agents as potential therapeutics to arrest amyloid formation, deposition, accumulation and/or persistence that occurs in Alzheimer's disease, Parkinson's disease, type II diabetes, and other amyloidoses are desperately sought.

### SUMMARY

Provided herein are compounds and pharmaceutical compositions containing compounds having formula:

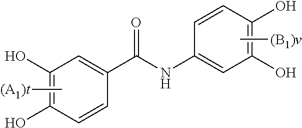

where A₁ and B₁ are each independently selected from hydrogen, halogen, cyanide, cyanate, thiocyanate, selenocyanate, trifluoromethoxy, azide, nitro, ⁺NH₃, SO₃H, carboxy and haloalkyl; and t and v are each independently 1 to 3.

In one embodiment, substitutents are appropriately selected to optimize physicochemical and/or biological properties such as, bioavailability, pharmacokinetics, blood-brain barrier penetration, optimized metabolism, and enhanced efficacy for treatment of amyloid diseases and synucleinopathies.

Also provided are any pharmaceutically-acceptable derivatives, including salts, esters, enol ethers or esters, acetals, ketals, orthoesters, hemiacetals, hemiketals, solvates, hydrates or prodrugs of the compounds. Pharmaceutically-acceptable salts, include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine, tris(hydroxymethyl)aminomethane, alkali metal salts, such as but not limited to lithium, potassium and sodium, alkali earth metal salts, such as but not limited to barium, calcium and magnesium, transition metal salts, such as but not limited to zinc and other metal salts, such as but not limited to sodium hydrogen phosphate and disodium phosphate, and also including, but not limited to, salts of mineral acids, such as but not limited to hydrochlorides and sulfates, salts of organic acids, such as but not limited to acetates, lactates, malates, tartrates, citrates, ascorbates, succinates, butyrates, valerates and fumarates.

Pharmaceutical formulations for administration by an appropriate route and means containing effective concentrations of one or more of the compounds provided herein or pharmaceutically acceptable derivatives, such as salts, esters, enol ethers or esters, acetals, ketals, orthoesters, hemiacetals, hemiketals, solvates, hydrates or prodrugs, of the compounds that deliver amounts effective for the treatment of amyloid diseases, are also provided.

The formulations are compositions suitable for administration by any desired route and include solutions, suspensions, emulsions, tablets, dispersible tablets, pills, capsules, powders, dry powders for inhalation, sustained release formulations, aerosols for nasal and respiratory delivery, patches for transdermal delivery and any other suitable route. The compositions should be suitable for oral administration, parenteral administration by injection, including subcutaneously, intramuscularly or intravenously as an injectable aqueous or oily solution or emulsion, transdermal administration and other selected routes.

Methods using such compounds and compositions for disrupting, disaggregating and causing removal, reduction or clearance of amyloid or synuclein fibrils are provided thereby providing new treatments for amyloid diseases and synucleinopathies.

Also provided are methods for treatment, prevention or amelioration of one or more symptoms of amyloid diseases or amyloidoses, including but not limited to diseases associated with the formation, deposition, accumulation, or persistence of amyloid fibrils, for example, the fibrils of an amyloid protein selected from Aβ amyloid, AA amyloid, AL amyloid, IAPP amyloid, PrP amyloid, α₂-microglobulin amyloid, transthyretin, prealbumin, and procalcitonin.

Methods for treatment of amyloid diseases, include, but are not limited to Alzheimer's disease, Down's syndrome, dementia pugilistica, multiple system atrophy, inclusion body myositosis, hereditary cerebral hemorrhage with amyloidosis of the Dutch type, Nieman-Pick disease type C, cerebral β-amyloid angiopathy, dementia associated with cortical basal degeneration, the amyloidosis of type 2 diabetes, the amyloidosis of chronic inflammation, the amyloidosis of malignancy and Familial Mediterranean Fever, the amyloidosis of multiple myeloma and B-cell dyscrasias, the amyloidosis of the prion diseases, Creutzfeldt-Jakob disease, Gerstmann-Straussler syndrome, kuru, scrapie, the amyloidosis associated with carpal tunnel syndrome, senile cardiac amyloidosis, familial amyloidotic polyneuropathy, and the amyloidosis associated with endocrine tumors.

Also provided are methods for treatment, prevention or amelioration of one or more symptoms of synuclein diseases or synucleinopathies. In one embodiment, the methods inhibit or prevent α-synuclein/NAC fibril formation, inhibit or prevent α-synuclein/NAC fibril growth, and/or cause disassembly, disruption, and/or disaggregation of preformed α-synuclein/NAC fibrils and α-synuclein/NAC-associated protein deposits. Synuclein diseases include, but are not limited to Parkinson's disease, familial Parkinson's disease, Lewy body disease, the Lewy body variant of Alzheimer's disease, dementia with Lewy bodies, multiple system atrophy, and the Parkinsonism-dementia complex of Guam.

### DETAILED DESCRIPTION

### A. Definitions

Unless defined otherwise, all technical and scientific terms used herein have the same meaning as is commonly understood by one of ordinary skill in the art to which this invention belongs. All patents, applications, published applications and other publications are incorporated by reference in their entirety. In the event that there are a plurality of definitions for a term herein, those in this section prevail unless stated otherwise.

As used herein “Amyloid diseases” or “amyloidoses” are diseases associated with the formation, deposition, accumulation, or persistence of amyloid fibrils, including but not limited to the fibrils of an amyloid protein selected from Aβ amyloid, AA amyloid, AL amyloid, IAPP amyloid, PrP amyloid, α₂-microglobulin amyloid, transthyretin, prealbumin, and procalcitonin. Such diseases include, but are not limited to Alzheimer's disease, Down's syndrome, dementia pugilistica, multiple system atrophy, inclusion body myositosis, hereditary cerebral hemorrhage with amyloidosis of the Dutch type, Nieman-Pick disease type C, cerebral β-amyloid angiopathy, dementia associated with cortical basal degeneration, the amyloidosis of type 2 diabetes, the amyloidosis of chronic inflammation, the amyloidosis of malignancy and Familial Mediterranean Fever, the amyloidosis of multiple myeloma and B-cell dyscrasias, the amyloidosis of the prion diseases, Creutzfeldt-Jakob disease, Gerstmann-Straussler syndrome, kuru, scrapie, the amyloidosis associated with carpal tunnel syndrome, senile cardiac amyloidosis, familial amyloidotic polyneuropathy, and the amyloidosis associated with endocrine tumors.

As used herein, “Synuclein diseases” or “synucleinopathies” are diseases associated with the formation, deposition, accumulation, or persistence of synuclein fibrils, including, but not limited to α-synuclein fibrils. Such diseases include, but are not limited to Parkinson's disease, familial Parkinson's disease, Lewy body disease, the Lewy body variant of Alzheimer's disease, dementia with Lewy bodies, multiple system atrophy, and the Parkinsonism-dementia complex of Guam.

“Fibrillogenesis” refers to the formation, deposition, accumulation and/or persistence of amyloid fibrils, filaments, inclusions, deposits, as well as synuclein (usually involving α-synuclein) and/or NAC fibrils, filaments, inclusions, deposits or the like.

“Inhibition of fibrillogenesis” refers to the inhibition of formation, deposition, accumulation and/or persistence of such amyloid fibrils or synuclein fibril-like deposits.

“Disruption of fibrils or fibrillogenesis” refers to the disruption of pre-formed amyloid or synuclein fibrils, that usually exist in a pre-dominant β-pleated sheet secondary structure. Such disruption by compounds provided herein may involve marked reduction or disassembly of amyloid or synuclein fibrils as assessed by various methods such as Thioflavin T fluorometry, Congo red binding, SDS-PAGE/Western blotting, as demonstrated by the Examples presented in this application.

“Mammal” includes both humans and non-human mammals, such as companion animals (cats, dogs, and the like), laboratory animals (such as mice, rats, guinea pigs, and the like) and farm animals (cattle, horses, sheep, goats, swine, and the like).

“Pharmaceutically acceptable excipient” means an excipient that is conventionally useful in preparing a pharmaceutical composition that is generally safe, non-toxic, and desirable, and includes excipients that are acceptable for veterinary use or for human pharmaceutical use. Such excipients may be solid, liquid, semisolid, or, in the case of an aerosol composition, gaseous.

A “therapeutically effective amount” means the amount that, when administered to a subject or animal for treating a disease, is sufficient to affect the desired degree of treatment, prevention or symptom amelioration for the disease. A “therapeutically effective amount” or a “therapeutically effective dosage” in certain embodiments inhibits, reduces, disrupts, disassembles amyloid or synuclein fibril formation, deposition, accumulation and/or persistence, or treats, prevents, or ameliorates one or more symptoms of a disease associated with these conditions, such as an amyloid disease or a synucleinopathy, in a measurable amount in one embodiment, by at least 20%, in other embodiment, by at least 40%, in other embodiment by at least 60%, and in still other embodiment by at least 80%, relative to an untreated subject. Effective amounts of a compound provided herein or composition thereof for treatment of a mammalian subject are about 0.1 to about 1000 mg/Kg of body weight of the subject/day, such as from about 1 to about 100 mg/Kg/day, in other embodiment, from about 10 to about 100 mg/Kg/day. A broad range of disclosed composition dosages are believed to be both safe and effective.

The term “sustained release component” is defined herein as a compound or compounds, including, but not limited to, polymers, polymer matrices, gels, permeable membranes, liposomes, microspheres, or the like, or a combination thereof, that facilitates the sustained release of the active ingredient.

If the complex is water-soluble, it may be formulated in an appropriate buffer, for example, phosphate buffered saline, or other physiologically compatible solutions. Alternatively, if the resulting complex has poor solubility in aqueous solvents, then it may be formulated with a non-ionic surfactant such as Tween, or polyethylene glycol. Thus, the compounds and their physiologically solvents may be formulated for administration by inhalation or insufflation (either through the mouth or the nose) or oral, buccal, parenteral, or rectal administration, as examples.

As used herein, pharmaceutically acceptable derivatives of a compound include salts, esters, enol ethers, enol esters, acetals, ketals, orthoesters, hemiacetals, hemiketals, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1″-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment of a disease also includes preventing the disease from occurring in a subject that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease), such as by disruption of pre-formed amyloid or synuclein fibrils. One such preventive treatment may be use of the disclosed compounds for the treatment of Mild Cognitive impairment (MCI).

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, “NAC” (non-Aβ component) is a 35-amino acid peptide fragment of α-synuclein, which like α-synuclein, has the ability to form amyloid-like fibrils when incubated at 37° C., and is positive with amyloid stains such as Congo red (demonstrating a red/green birefringence when viewed under polarized light) and Thioflavin S (demonstrating positive fluorescence) (Hashimoto et al., Brain Res. 799:301-306, 1998; Ueda et al., Proc. Natl. Acad. Sci. U.S.A. 90:11282-11286, 1993). Inhibition of NAC fibril formation, deposition, accumulation, aggregation, and/or persistence is believed to be effective treatment for a number of diseases involving α-synuclein, such as Parkinson's disease, Lewy body disease and multiple system atrophy.

As used herein, a prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. In the case of amino acid residues, such residues may be of either the L- or D-form. The configuration for naturally occurring amino acid residues is generally L. When not specified the residue is the L form. As used herein, the term “amino acid” refers to α-amino acids which are racemic, or of either the D- or L-configuration. The designation “d” preceding an amino acid designation (e.g., dAla, dSer, dVal, etc.) refers to the D-isomer of the amino acid. The designation “dl” preceding an amino acid designation (e.g., dlPip) refers to a mixture of the L- and D-isomers of the amino acid. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, alkyl, alkenyl and alkynyl carbon chains, if not specified, contain from 1 to 20 carbons, or 1 or 2 to 16 carbons, and are straight or branched. Alkenyl carbon chains of from 2 to 20 carbons, in certain embodiments, contain 1 to 8 double bonds and alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, allyl (propenyl) and propargyl (propynyl). As used herein, lower alkyl, lower alkenyl, and lower alkynyl refer to carbon chains having from about 1 or about 2 carbons up to about 6 carbons. As used herein, “alk(en)(yn)yl” refers to an alkyl group containing at least one double bond and at least one triple bond.

As used herein, “halo”, “halogen” or “halide” refers to F, Cl, Br or I.

As used herein, pseudohalides or pseudohalo groups are groups that behave substantially similar to halides. Such compounds can be used in the same mariner and treated in the same manner as halides. Pseudohalides include, but are not limited to, cyanide, cyanate, thiocyanate, selenocyanate, trifluoromethoxy, and azide.

As used herein, “haloalkyl” refers to an alkyl group in which one or more of the hydrogen atoms are replaced by halogen. Such groups include, but are not limited to, chloromethyl, trifluoromethyl and 1-chloro-2-fluoroethyl.

As used herein, “haloalkoxy” refers to RO— in which R is a haloalkyl group.

As used herein, “sulfinyl” or “thionyl” refers to —S(O)—. As used herein, “sulfonyl” or “sulfuryl” refers to —S(O)₂—. As used herein, “sulfo” refers to —S(O)₂O—.

As used herein, “carboxy” refers to a divalent radical, —C(O)O—.

As used herein, “aminocarbonyl” refers to —C(O)NH₂.

As used herein, “alkylaminocarbonyl” refers to —C(O)NHR in which R is alkyl, including lower alkyl. As used herein, “dialkylaminocarbonyl” refers to —C(O)NR′R in which R′ and R are each independently alkyl, including lower alkyl; “carboxamide” refers to groups of formula —NR′COR in which R′ and R are each independently alkyl, including lower alkyl.

As used herein, “arylalkylaminocarbonyl” refers to —C(O)NRR′ in which one of R and R′ is aryl, including lower aryl, such as phenyl, and the other of R and R′ is alkyl, including lower alkyl.

As used herein, “arylaminocarbonyl” refers to —C(O)NHR in which R is aryl, including lower aryl, such as phenyl.

As used herein, “hydroxycarbonyl” refers to —COOH.

As used herein, “alkoxycarbonyl” refers to —C(O)OR in which R is alkyl, including lower alkyl.

As used herein, “aryloxycarbonyl” refers to —C(O)OR in which R is aryl, including lower aryl, such as phenyl.

As used herein, “alkoxy” and “alkylthio” refer to RO— and RS—, in which R is alkyl, including lower alkyl.

As used herein, “aryloxy” and “arylthio” refer to RO— and RS—, in which R is aryl, including lower aryl, such as phenyl.

Where the number of any given substituent is not specified (e.g., haloalkyl), there may be one or more substituents present. For example, “haloalkyl” may include one or more of the same or different halogens. As another example, “C₁₋₃alkoxyphenyl” may include one or more of the same or different alkoxy groups containing one, two or three carbons.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

### B. Compounds

Provided herein are compounds and pharmaceutical compositions containing compounds having formula:

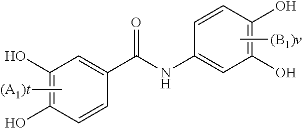

or a pharmaceutically acceptable salt thereof,

In other embodiments, the compound has the formula:

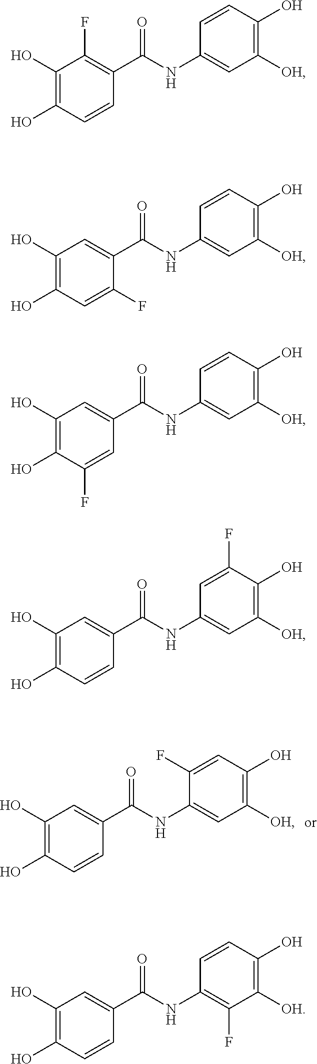

In one embodiment, the compound is selected from a group of 3,4,3′,4′-tetrahydroxybenzoin; 3,4,3′,4′-tetrahydroxydesoxybenzoin; 3,4,3′,4′-tetrahydroxydiphenylmethane; 1,2-bis(3,4-dihydroxyphenyl)ethane; 1,3-bis(3,4-dihydroxyphenyl)propane; 3,4,3′,4′-tetrahydroxychalcone; 3,5-bis(3,4-dihydroxyphenyl)-1-methyl-2-pyrazoline; 4,6-bis(3,4-dihydroxyphenyl)-3-cyano-2-methylpyridine; 1,4-bis(3,4-dihydroxybenzyl)piperazine; N,N′-bis(3,4-dihydroxybenzyl)-N,N′-dimethyl-ethylenediamine; 2,5-bis(3,4-dihydroxybenzyl)-2,5-diaza[2.2.1]bicycloheptane; bis(3,4-dihydroxybenzyl)-trans-1,2-diaminocyclohexane; N,N′-bis(3,4-dihydroxybenzyl)-trans-1,4-diaminocyclohexane; N,N′-bis(3,4-dihydroxybenzyl)-cis-1,3-bis(aminomethyl)cyclohexane; N-(3,4-dihydroxybenzyl)proline 3,4-dihydroxybenzylamide; 2-(3,4-dihydroxybenzyl)isoquinoline-3-carboxylic acid 3,4-dihydroxyphenethylamide; 2,6-bis(3,4-dihydroxybenzyl)cyclohexanone; 3,5-bis(3,4-dihydroxybenzyl)-1-methyl-4-piperidinone; 2,4-bis(3,4-dihydroxybenzyl)-3-tropinone; tris(3,4-dihydroxybenzyl)methane; α-(3,4-dihydroxybenzamido)-3,4-dihydroxycinnamic acid 3,4-dihydroxybenzyl amide; 4-(3,4-dihydroxybenzylaminomethylene)-2-(3,4-dihydroxyphenyl)oxazolin-5-one; 1,4-bis(3,4-dihydroxybenzoyl)piperazine; N,N′-bis(3,4-dihydroxybenzoyl)-N,N′-dimethylethylenediamine; 2,5-bis(3,4-dihydroxybenzoyl)-2,5-diaza[2.2.1]bicycloheptane; N,N′-bis(3,4-dihydroxybenzoyl)-trans-1,2-diaminocyclohexane; N,N′-bis(3,4-dihydroxybenzoyl)-cis-1,3-bis(aminomethyl)cyclohexane; 3,6-bis(3,4-dihydroxybenzyl)-2,5-diketopiperazine; 3,6-bis(3,4-dihydroxybenzylidene)-1,4-dimethyl-2,5-diketopiperazine; N-(3,4-dihydroxyphenylacetyl)proline-3,4-dihydroxyanilide; 2,3-bis(3,4-dihydroxyphenyl)butane; 1,3-bis(3,4-dihydroxybenzyl)benzene; 1,4-bis(3,4-dihydroxybenzyl)benzene; 2,6-bis(3,4-dihydroxybenzyl)pyridine; 2,5-bis(3,4-dihydroxybenzyl)thiophene; 2,3-bis(3,4-dihydroxybenzyl)thiophene; 1,2-bis(3,4-dihydroxyphenyl)cyclohexane; 1,4-bis(3,4-dihydroxyphenyl)cyclohexane; 3,7-bis(3,4-dihydroxyphenyl)bicyclo[3.3.0]octane; 2,3-bis(3,4-dihydroxyphenyl)-1,7,7-trimethyl-bicyclo[2.2.1]heptane; 1,2-bis(3,4-dihydroxyphenoxy)ethane; 1,3-bis(3,4-dihydroxyphenoxy)propane; trans-1,2-bis(3,4-dihydroxyphenoxy)cyclopentane; N-(3,4-dihydroxybenzyl)-3-(3,4-dihydroxyphenoxy)-2-hydroxypropylamine; 3,4-dihydroxyphenoxyacetic acid 3,4-dihydroxyanilide; 3,4-dihydroxyphenoxyacetic acid 3,4-dihydroxybenzylamide; 3,4-dihydroxyphenoxyacetic acid 3,4-dihydroxyphenethylamide; 3,4-dihydroxybenzoic acid p-(3,4-dihydroxyphenoxy)anilide; 3,4-dihydroxybenzoic acid o-(3,4-dihydroxyphenoxy)anilide; 2,6-bis(3,4-dihydroxyphenoxy)pyridine; 3,4-dihydroxybenzoic acid 3,4-dihydroxyanilide; 3,4-dihydroxybenzoic acid 3,4-dihydroxybenzylamide; 3,4-dihydroxybenzoic acid 3,4-dihydroxyphenethylamide; 3,4-dihydroxyphenyl acetic acid 3,4-dihydroxyanilide; 3,4-dihydroxyphenylacetic acid 3,4-dihydroxybenzylamide; 3,4-dihydroxyphenylacetic acid 3,4-dihydroxyphenethylamide; dihydroxyphenyl)propionic acid 3,4-dihydroxyanilide; 3-(3,4-dihydroxyphenyl)propionic acid 3,4-dihydroxybenzylamide; 3-(3,4-dihydroxyphenyl)propionic acid 3,4-dihydroxyphenethylamide; 3,4-dihydroxycinnamic acid 3,4-dihydroxyanilide; 3,4-dihydroxycinnamic acid 3,4-dihydroxybenzylamide; 3,4-dihydroxycinnamic acid 3,4-dihydroxyphenethylamide; oxalic acid bis(3,4-dihydroxyanilide); oxalic acid bis(3,4-dihydroxybenzylamide); oxalic acid bis(3,4-dihydroxyphenethylamide); succinic acid bis(3,4-dihydroxyanilide); succinic acid bis(3,4-dihydroxybenzylamide); succinic acid bis(3,4-dihydroxyphenethylamide); maleic acid bis(3,4-dihydroxyanilide); maleic acid bis(3,4-dihydroxybenzylamide); fumaric acid bis(3,4-dihydroxyanilide); fumaric acid bis(3,4-dihydroxybenzylamide); bis(3,4-dihydroxybenzyl)amine; N-(3,4-dihydroxy-benzyl)-3,4-dihydroxyphenethylamine; tris(3,4-dihydroxybenzyl)amine; 1,3-bis(3,4-dihydroxyphenyl)urea; 1-(3,4-dihydroxyphenyl)-3-(3,4-dihydroxybenzyl)urea; 1-(3,4-dihydroxyphenyl)-3-(3,4-dihydroxyphenethyl)urea; 3-deoxy-3-(3,4-dihydroxybenzyl)aminoepicatechin; 3-deoxy-3-(3,4-dihydroxyphenethyl)-aminoepicatechin; 2,3,6,7-tetrahydroxy-9,10-epoxy-9,10-dihydroacridine; 10-aminoanthracene-1,2,7,8-tetraol; acridine-1,2,6,7-tetraol; phenoxazine-2,3,7,8,10-pentaol; dibenzo[c,f][2,7]napthyridine-2,3,10,11-tetraol; and 6-methyl-5,6,6a,7-tetrahydro-4H-dibenzo[de,g]quinoline-2,10,11-triol, wherein at least one of the phenolic hydroxy groups of the compound is replaced.

In certain embodiments, the compound is selected from

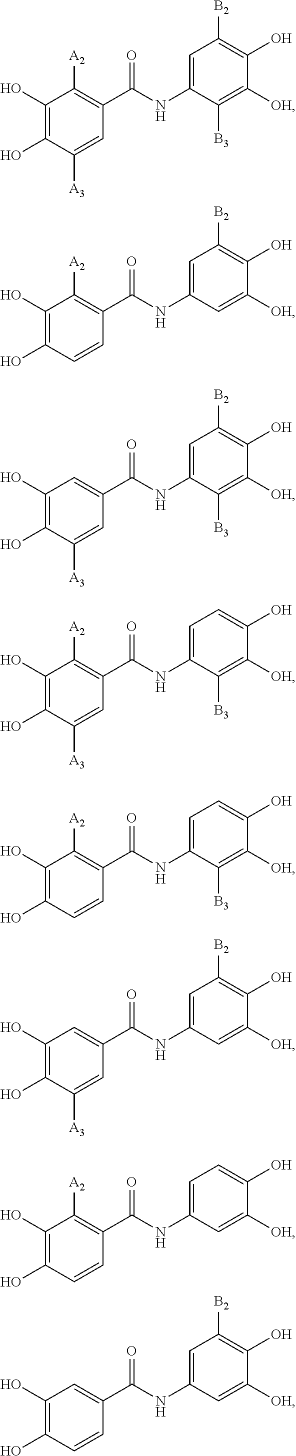

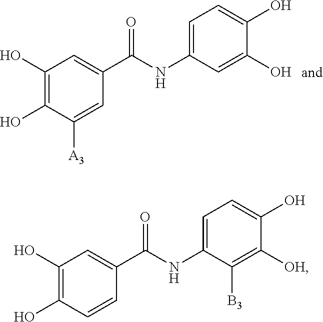

wherein A₂, A₃, B₂ and B₃ are each independently selected from Cl, F, cyanide, cyanate, thiocyanate, selenocyanate, trifluoromethoxy, azide, nitro and trifluoromethyl.

### C. Preparation of the Compounds

The compounds provided herein can be prepared by standard synthetic methods known in the art, and are shown in general schemes provided herein. The examples that follow describe the exemplary embodiments and are not purported to limit the scope of the claimed subject matter. It is intended that the specification, together with the following examples, be considered exemplary only, with the scope and spirit of the claimed subject matter being indicated by the claims that follow these examples. Other embodiments within the scope of claims herein will be apparent to one skilled in the art from consideration of the specification as described herein.

The starting materials and reagents used in preparing these compounds are either available from commercial suppliers such as the Aldrich Chemical Company (Milwaukee, Wis.), Bachem (Torrance, Calif.), Sigma (St. Louis, Mo.), or Lancaster Synthesis Inc. (Windham, N.H.) or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

In most cases, protective groups for the hydroxy groups are introduced and finally removed. Suitable protective groups are described in Greene et al., Protective Groups in Organic Synthesis, Second Edition, John Wiley and Sons, New York, 1991. Other starting materials or early intermediates may be prepared by elaboration of the materials listed above, for example, by methods well known to a person of ordinary skill in the art. The starting materials, intermediates, and compounds provided herein may be isolated and purified using conventional techniques, including precipitation, filtration, distillation, crystallization, chromatography, and the like. The compounds may be characterized using conventional methods, including physical constants and spectroscopic methods.

Provided herein are general reaction schemes for the preparation of exemplary compounds.

i) Achesom et al. J. Med. Chem. (1981) 24, 1300-1304, describe use of thionyl chloride for preparing benxthiadiazolidine S,S-dioxide as follows:

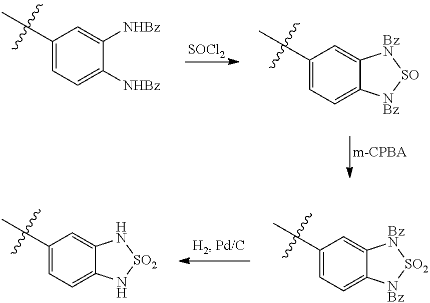

ii) Preparation of nitro benzothiadiazolidine S,S-dioxide is described by Burke et al. in JCS Perkin Transactions (1984) 11, 1851-4, as follows:

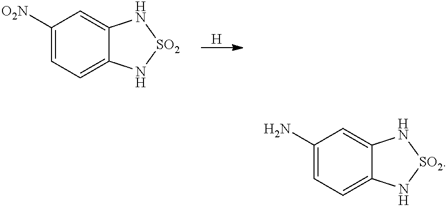

Further compounds provided herein can be prepared by reactions described in the literature as follows:

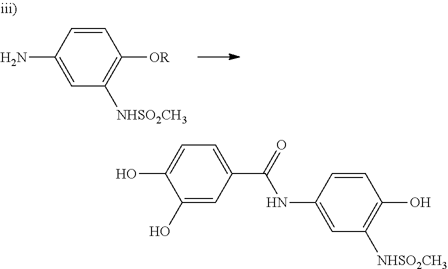

See, Roberts et al., J.O. Chen. (1997) 62, 568-577

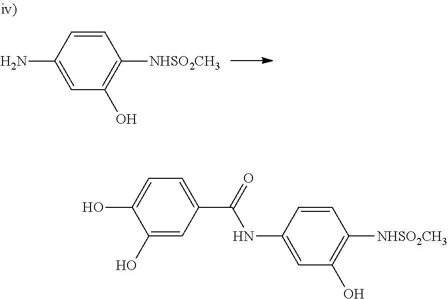

See, Hughes et al., J. Med. Chem. (1957) 18, 1077-1088.

### D. Pharmaceutical Compositions and Administration

The compounds provided herein can be used as such, be administered in the form of pharmaceutically acceptable salts derived from inorganic or organic acids, or used in combination with one or more pharmaceutically acceptable excipients. The phrase “pharmaceutically acceptable salt” means those salts which are, within the scope of sound medical judgment, suitable for use in contact with the tissues without undue toxicity, irritation, allergic response, and the like, and are commensurate with a reasonable benefit/risk ratio. Pharmaceutically acceptable salts are well known in the art. The salts can be prepared either in situ during the final isolation and purification of the compounds provided herein or separately by reacting the acidic or basic drug substance with a suitable base or acid respectively. Typical salts derived from organic or inorganic acids salts include, but are not limited to hydrochloride, hydrobromide, hydroiodide, acetate, adipate, alginate, citrate, aspartate, benzoate, bisulfate, gluconate, fumarase, hydroiodide, lactate, maleate, oxalate, palmitoate, pectinate, succinate, tartrate, phosphate, glutamate, and bicarbonate. Typical salts derived from organic or inorganic bases include, but are not limited to lithium, sodium, potassium, calcium, magnesium, ammonium, monoalkylammonium such as meglumine, dialkylammonium, trialkylammonium, and tetralkylammonium.

In certain embodiments, the compositions contain a compound provided herein that is at least substantially pure. In general “pure” means better than 95% pure, and “substantially pure” means a compound synthesized such that the compound, as made as available for consideration into a therapeutic dosage, has only those impurities that can not readily nor reasonably be removed by conventional purification processes.

The mode of administration of the pharmaceutical compositions can be oral, rectal, intravenous, intramuscular, intracisternal, intravaginal, intraperitoneal, bucal, subcutaneous, intrasternal, nasal, or topical. The compositions can also be delivered at the target site through a catheter, an intracoronary stent (a tubular device composed of a fine wire mesh), a biodegradable polymer, or biological carriers including, but are not limited to antibodies, biotin-avidin complexes, and the like. Dosage forms for topical administration of a compound provided herein include powders, sprays, ointments and inhalants. The active compound is mixed under sterile conditions with a pharmaceutically acceptable carrier and any needed preservatives, buffers or propellants. Opthalmic formulations, eye ointments, powders and solutions are also provided herein.

Actual dosage levels of active ingredients and the mode of administration of the pharmaceutical compositions provided herein can be varied in order to achieve the effective therapeutic response for a particular patient. The phrase “therapeutically effective amount” of the compound provided herein means a sufficient amount of the compound to treat disorders, at a reasonable benefit/risk ratio applicable to any medical treatment. It will be understood, however, that the total daily usage of the compounds and compositions of the provided will be decided by the attending physician within the scope of sound medical judgment. The total daily dose of the compounds provided herein may range from about 0.0001 to about 1000 mg/kg/day. For purposes of oral administration, doses can be in the range from about 0.001 to about 5 mg/kg/day. If desired, the effective daily dose can be divided into multiple doses for purposes of administration; consequently, single dose compositions may contain such amounts or submultiples thereof to make up the daily dose. The specific therapeutically effective dose level for any particular patient will depend upon a variety of factors including the disorder being treated and the severity of the disorder; medical history of the patient, activity of the specific compound employed; the specific composition employed, age, body weight, general health, sex and diet of the patient, the time of administration, route of administration, the duration of the treatment, rate of excretion of the specific compound employed, drugs used in combination or coincidental with the specific compound employed; and the like.

The compounds provided can be formulated together with one or more non-toxic pharmaceutically acceptable diluents, carriers, adjuvants, and antibacterial and antifungal agents such as parabens, chlorobutanol, phenol, sorbic acid, and the like. Proper fluidity can be maintained, for example, by the use of coating materials such as lecithin, by the maintenance of the required particle size in the case of dispersions, and by the use of surfactants. In some cases, in order to prolong the effect of the drug, it is desirable to decrease the rate of absorption of the drug from subcutaneous or intramuscular injection. This can be accomplished by suspending crystalline or amorphous drug substance in a vehicle having poor water solubility such as oils. The rate of absorption of the drug then depends upon its rate of dissolution, which, in turn, may depend upon crystal size and crystalline form. Prolonged absorption of an injectable pharmaceutical form can be achieved by the use of absorption delaying agents such as aluminum monostearate or gelatin.

The compound provided herein can be administered enterally or parenterally in solid or liquid forms. Compositions suitable for parenteral injection may comprise physiologically acceptable, isotonic sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), vegetable oils (such as olive oil), injectable organic esters such as ethyl oleate, and suitable mixtures thereof. These compositions can also contain adjuvants such as preserving, wetting, emulsifying, and dispensing agents. Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar and tragacanth, or mixtures of these substances.

The compounds provided herein can also be administered by injection or infusion, either subcutaneously or intravenously, or intramuscularly, or intrasternally, or intranasally, or by infusion techniques in the form of sterile injectable or oleaginous suspension. The compound may be in the form of a sterile injectable aqueous or oleaginous suspensions. These suspensions may be formulated according to the known art using suitable dispersing of wetting agents and suspending agents that have been described above. The sterile injectable preparation may also be a sterile injectable solution or suspension in a non-toxic parenterally-acceptable diluent or solvent for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oils may be conventionally employed including synthetic mono- or diglycerides. In addition fatty acids such as oleic acid find use in the preparation of injectables. Dosage regimens can be adjusted to provide the optimum therapeutic response. For example, several divided dosages may be administered daily or the dosage may be proportionally reduced as indicated by the exigencies of the therapeutic situation.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or (a) fillers or extenders such as starches, lactose, sucrose, glucose, mannitol and silicic acid; (b) binders such as carboxymethylcellulose, alginates, gelatin, polyvinylpyrrolidone, sucrose and acacia; (c) humectants such as glycerol; (d) disintegrating agents such as agar-agar, calcium carbonate, potato or tapioca starch, alginic acid, certain silicates and sodium carbonate; (e) solution retarding agents such as paraffin; (f) absorption accelerators such as quaternary ammonium compounds; (g) wetting agents such as cetyl alcohol and glycerol monostearate; (h) absorbents such as kaolin and bentonite clay and (i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes. Tablets contain the compound in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, maize starch or alginic acid; binding agents, for example, maize starch, gelatin or acacia, and lubricating agents, for example, magnesium stearate or stearic acid or tale. The tablets may be uncoated or they may be coated by known techniques to delay disintegration and absorption in the gastrointestinal tract and thereby provide a sustained action over a longer period. For example, a time delay material such as glycerol monostearate or glycerol distearate may be employed. Formulations for oral use may also be presented as hard gelatin capsules wherein the compound is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavoring and perfuming agents.

Aqueous suspensions contain the compound in admixture with excipients suitable for the manufacture of aqueous suspensions. Such excipients are suspending agents, for example, sodium carboxymethylcellulose, methylcellulose, hydroxypropylmethyl cellulose, sodium alginate, polyvinylpyrrolidone, gum tragacanth and gum acacia; dispersing or wetting agents may be naturally occurring phosphatides, for example lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids such as hexitol such as polyoxyethylene sorbitol monooleate, or condensation products of ethylene oxide with partial esters from fatty acids and a hexitol anhydrides, for example, polyethylene sorbitan monooleate. The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the compound in a vegetable oil, for example arachis oil, olive oil, sesame oil, or coconut oil or in a mineral oil such as liquid paraffin. The oily suspensions may contain a thickening agent, for example beeswax, hard paraffin or cetyl alcohol. Sweetening agents, such as those set forth below, and flavoring agents may be added to provide a palatable oral preparation. These compositions may be preserved by the addition of an antioxidant such as ascorbic acid. Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already described above. Additional excipients, for example sweetening, flavoring and agents, may also be present.

The compounds provided herein may also be in the form of oil-in-water emulsions. The oily phase may be a vegetable oil, for example olive oil or arachis oils, or a mineral oil, for example liquid paraffin or mixtures of these. Suitable emulsifying agents may be naturally-occurring gums, for example gum acacia or gum tragacanth, naturally occurring phosphatides, for example soy bean, lecithin, and occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsion may also contain sweetening and flavoring agents. Syrups and elixirs may be formulated with sweetening agents, for example, glycerol, sorbitol or sucrose. Such formulations may also contain a demulcent, a preservative and flavoring and coloring agents.

In one embodiment, the compounds are formulated in dosage unit form for ease of administration and uniformity of dosage. Dosage unit form as used herein refers to physically discrete units suited as unitary dosages for the subjects to be treated; each containing a therapeutically effective quantity of the compound and at least one pharmaceutical excipient. A drug product will comprise a dosage unit form within a container that is labeled or accompanied by a label indicating the intended method of treatment, such as the treatment of an amyloid disease, for example an amyloidosis such as Alzheimer's disease or a disease associated with α-synuclein/NAC fibril formation such as Parkinson's disease. Compositions for rectal or vaginal administration are preferably suppositories which can be prepared by mixing the compounds provided herein with suitable non-irritating excipients or carriers such as cocoa butter, polyethylene glycol or a suppository wax which are solid at room temperature but liquid at body temperature and therefore melt in the rectum or vaginal cavity and release the active compound.

Compounds provided herein can also be administered in the form of liposomes. Methods to form liposomes are known in the art (Prescott, Ed., Methods in Cell Biology 1976, Volume XIV, Academic Press, New York, N.Y.) As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound provided herein, stabilizers, preservatives, excipients and the like. The preferred lipids are natural and synthetic phospholipids and phosphatidyl cholines (lecithins).

The compounds provided herein can also be administered in the form of a ‘prodrug’ wherein the active pharmaceutical ingredients, represented by Formulas I-3, are released in vivo upon contact with hydrolytic enzymes such as esterases and phophatases in the body. The term “pharmaceutically acceptable prodrugs” as used herein represents those prodrugs of the compounds provided herein, which are, within the scope of sound medical judgment, suitable for use in contact with the tissues without undue toxicity, irritation, allergic response, and the like, commensurate with a reasonable benefit/risk ratio, and effective for their intended use. A thorough discussion is provided in T. Higuchi and V. Stella (Higuchi, T. and Stella, V. Pro-drugs as Novel Delivery Systems, V. 14 of the A.C.S. Symposium Series; Edward B. Roche, Ed., Bioreversible Carriers in Drug Design 1987, American Pharmaceutical Association and Pergamon Press), which is incorporated herein by reference.

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

Article of Manufacture

The compounds or pharmaceutically acceptable derivatives may be packaged as articles of manufacture containing packaging material, a compound or pharmaceutically acceptable derivative thereof provided herein, which is effective for treatment, prevention or amelioration of one or more symptoms of amyloidosis and synuclein diseases, within the packaging material, and a label that indicates that the compound or composition, or pharmaceutically acceptable derivative thereof, is used for treatment, prevention or amelioration of one or more symptoms of amyloidosis and synuclein diseases.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for amyloidosis and synuclein diseases.

Sustained Release Formulations

Also provided are sustained release formulations to deliver the compounds to the desired target (i.e. brain or systemic organs) at high circulating levels (between 10⁻⁹ and 10⁻⁴ M). In a certain embodiment for the treatment of Alzheimer's or Parkinson's disease, the circulating levels of the compounds is maintained up to 10⁻⁷ M. The levels are either circulating in the patient systemically, or in one embodiment, present in brain tissue, and in a another embodiments, localized to the amyloid or α-synuclein fibril deposits in brain or other tissues.

It is understood that the compound levels are maintained over a certain period of time as is desired and can be easily determined by one skilled in the art. In one embodiment, the administration of a sustained release formulation is effected so that a constant level of therapeutic compound is maintained between 10⁻⁸ and 10−6 M between 48 to 96 hours in the sera.

Such sustained and/or timed release formulations may be made by sustained release means of delivery devices that are well known to those of ordinary skill in the art, such as those described in U.S. Pat. Nos. 3,845,770; 3,916,899; 3,536,809; 3,598,123; 4,008,719; 4,710,384; 5,674,533; 5,059,595; 5,591,767; 5,120,548; 5,073,543; 5,639,476; 5,354,556 and 5,733,566, the disclosures of which are each incorporated herein by reference. These pharmaceutical compositions can be used to provide slow or sustained release of one or more of the active compounds using, for example, hydroxypropylmethyl cellulose, other polymer matrices, gels, permeable membranes, osmotic systems, multilayer coatings, microparticles, liposomes, microspheres, or the like. Suitable sustained release formulations known to those skilled in the art, including those described herein, may be readily selected for use with the pharmaceutical compositions provided herein. Thus, single unit dosage forms suitable for oral administration, such as, but not limited to, tablets, capsules, gelcaps, caplets, powders and the like, that are adapted for sustained release are contemplated herein.

In one embodiment, the sustained release formulation contains active compound such as, but not limited to, microcrystalline cellulose, maltodextrin, ethylcellulose, and magnesium stearate. As described above, all known methods for encapsulation which are compatible with properties of the disclosed compounds are contemplated herein. The sustained release formulation is encapsulated by coating particles or granules of the pharmaceutical compositions provided herein with varying thickness of slowly soluble polymers or by microencapsulation. In one embodiment, the sustained release formulation is encapsulated with a coating material of varying thickness (e.g. about 1 micron to 200 microns) that allow the dissolution of the pharmaceutical composition about 48 hours to about 72 hours after administration to a mammal. In another embodiment, the coating material is a food-approved additive.

In another embodiment, the sustained release formulation is a matrix dissolution device that is prepared by compressing the drug with a slowly soluble polymer carrier into a tablet. In one embodiment, the coated particles have a size range between about 0.1 to about 300 microns, as disclosed in U.S. Pat. Nos. 4,710,384 and 5,354,556, which are incorporated herein by reference in their entireties. Each of the particles is in the form of a micromatrix, with the active ingredient uniformly distributed throughout the polymer.

Sustained release formulations such as those described in U.S. Pat. No. 4,710,384, which is incorporated herein by reference in its entirety, having a relatively high percentage of plasticizer in the coating in order to permit sufficient flexibility to prevent substantial breakage during compression are disclosed. The specific amount of plasticizer varies depending on the nature of the coating and the particular plasticizer used. The amount may be readily determined empirically by testing the release characteristics of the tablets formed. If the medicament is released too quickly, then more plasticizer is used. Release characteristics are also a function of the thickness of the coating. When substantial amounts of plasticizer are used, the sustained release capacity of the coating diminishes. Thus, the thickness of the coating may be increased slightly to make up for an increase in the amount of plasticizer. Generally, the plasticizer in such an embodiment will be present in an amount of about 15 to 30% of the sustained release material in the coating, in one embodiment 20 to 25%, and the amount of coating will be from 10 to 25% of the weight of the active material, and in another embodiment, 15 to 20% of the weight of active material. Any conventional pharmaceutically acceptable plasticizer may be incorporated into the coating.

The compounds provided herein can be formulated as a sustained and/or timed release formulation. All sustained release pharmaceutical products have a common goal of improving drug therapy over that achieved by their non-sustained counterparts. Ideally, the use of an optimally designed sustained release preparation in medical treatment is characterized by a minimum of drug substance being employed to cure or control the condition. Advantages of sustained release formulations may include: 1) extended activity of the composition, 2) reduced dosage frequency, and 3) increased patient compliance. In addition, sustained release formulations can be used to affect the time of onset of action or other characteristics, such as blood levels of the composition, and thus can affect the occurrence of side effects.

The sustained release formulations provided herein are designed to initially release an amount of the therapeutic composition that promptly produces the desired therapeutic effect, and gradually and continually release of other amounts of compositions to maintain this level of therapeutic effect over an extended period of time. In order to maintain this constant level in the body, the therapeutic composition must be released from the dosage form at a rate that will replace the composition being metabolized and excreted from the body.

The sustained release of an active ingredient may be stimulated by various inducers, for example pH, temperature, enzymes, water, or other physiological conditions or compounds.

Preparations for oral administration may be suitably formulated to give controlled release of the active compound. In one embodiment, the compounds are formulated as controlled release powders of discrete microparticles that can be readily formulated in liquid form. The sustained release powder comprises particles containing an active ingredient and optionally, an excipient with at least one non-toxic polymer.

The powder can be dispersed or suspended in a liquid vehicle and will maintain its sustained release characteristics for a useful period of time. These dispersions or suspensions have both chemical stability and stability in terms of dissolution rate. The powder may contain an excipient comprising a polymer, which may be soluble, insoluble, permeable, impermeable, or biodegradable. The polymers may be polymers or copolymers. The polymer may be a natural or synthetic polymer. Natural polymers include polypeptides (e.g., zein), polysaccharides (e.g., cellulose), and alginic acid. Representative synthetic polymers include those described, but not limited to, those described in column 3, lines 33-45 of U.S. Pat. No. 5,354,556, which is incorporated by reference in its entirety. Particularly suitable polymers include those described, but not limited to those described in column 3, line 46-column 4, line 8 of U.S. Pat. No. 5,354,556 which is incorporated by reference in its entirety.

The sustained release compositions provided herein may be formulated for parenteral administration, e.g., by intramuscular injections or implants for subcutaneous tissues and various body cavities and transdermal devices. In one embodiment, intramuscular injections are formulated as aqueous or oil suspensions. In an aqueous suspension, the sustained release effect is due to, in part, a reduction in solubility of the active compound upon complexation or a decrease in dissolution rate. A similar approach is taken with oil suspensions and solutions, wherein the release rate of an active compound is determined by partitioning of the active compound out of the oil into the surrounding aqueous medium. Only active compounds which are oil soluble and have the desired partition characteristics are suitable. Oils that may be used for intramuscular injection include, but are not limited to, sesame, olive, arachis, maize, almond, soybean, cottonseed and castor oil.

A highly developed form of drug delivery that imparts sustained release over periods of time ranging from days to years is to implant a drug-bearing polymeric device subcutaneously or in various body cavities. The polymer material used in an implant, which must be biocompatible and nontoxic, include but are not limited to hydrogels, silicones, polyethylenes, ethylene-vinyl acetate copolymers, or biodegradable polymers.

### E. Evaluation of the Activity of the Compounds

The biological activity of the compounds provided herein as disruptors/inhibitors of Alzheimer's disease β-amyloid protein (Aβ) fibrils, type 2 diabetes IAPP fibrils and Parkinson's disease NAC fibrils was assessed by determining the efficacy of the compounds to cause a disassembly/disruption of pre-formed amyloid fibrils of Alzheimer's disease (i.e. consisting of Aβ 1-42 fibrils), IAPP fibrils and Parkinson's disease NAC fibrils. In one study, Thioflavin T fluorometry was used to determine the effects of the compounds, and of EDTA (as a negative control). In this assay Thioflavin T binds specifically to fibrillar amyloid, and this binding produces a fluorescence enhancement at 485 nm that is directly proportional to the amount of fibrils present. The higher the fluorescence, the greater the amount of fibrils present (Naki et al, Lab. Invest. 65:104-110, 1991; Levine III, Protein Sci. 2:404-410, 1993; Amyloid: Int. J. Exp. Clin. Invest. 2:1-6, 1995). The disruption of Aβ 1-42, even in its monomeric form, was confirmed by a study involving the use of SDS-PAGE and Western blotting methods.

In the Congo red binding assay the ability of a given test compound to alter amyloid (Aβ 1-42 fibrils, IAPP fibrils or NAC fibrils) binding to Congo red was quantified. In this assay, Aβ 1-42 fibrils, IAPP fibril or NAC fibrils and test compounds were incubated for 3 days and then vacuum filtered through a 0.2 μm filter. The amount of Aβ 1-42 fibrils, IAPP fibrils or NAC fibrils retained in the filter was then quantitated following staining of the filter with Congo red. After appropriate washing of the filter, any lowering of the Congo red color on the filter in the presence of the test compound (compared to the Congo red staining of the amyloid protein in the absence of the test compound) was indicative of the test compound's ability to diminish/alter the amount of aggregated and congophilic Aβ 1-42 fibrils, IAPP fibrils or NAC fibrils.

### F. Combination Therapy

In another embodiment, the compounds may be administered in combination, or sequentially, with another therapeutic agent. Such other therapeutic agents include those known for treatment, prevention, or amelioration of one or more symptoms of amyloidosis and synuclein diseases. Such therapeutic agents include, but are not limited to, donepezil hydrochloride (Aracept), rivastigmine tartrate (Exelon), tacrine hydrochloride (Cognex) and galantamine hydrobromide (Reminyl).

### G. Methods of Use of the Compounds and Compositions

The compounds and compositions provided herein are useful in methods of treatment, prevention, or amelioration of one or more symptoms of amyloid diseases or disorders, including but not limited to diseases associated with the formation, deposition, accumulation, or persistence of amyloid fibrils diseases associated with the formation, deposition, accumulation, or persistence of amyloid fibrils. In one embodiment, the fibrils of an amyloid protein are selected from the group of Aβ amyloid, AA amyloid, AL amyloid, IAPP amyloid, PrP amyloid, α₂-microglobulin amyloid, transthyretin, prealbumin, and procalcitonin. In certain embodiments, the fibrils of an amyloid protein are Aβ amyloid and IAPP amyloid. In certain embodiments, the compounds and compositions provided herein are used for treatment, prevention, or amelioration of one or more symptoms of diseases including, but not limited to Alzheimer's disease, Down's syndrome, dementia pugilistica, multiple system atrophy, inclusion body myositosis, hereditary cerebral hemorrhage with amyloidosis of the Dutch type, Nieman-Pick disease type C, cerebral β-amyloid angiopathy, dementia associated with cortical basal degeneration, the amyloidosis of type 2 diabetes, the amyloidosis of chronic inflammation, the amyloidosis of malignancy and Familial Mediterranean Fever, the amyloidosis of multiple myeloma and B-cell dyscrasias, the amyloidosis of the prion diseases, Creutzfeldt-Jakob disease, Gerstmann-Straussler syndrome, kuru, scrapie, the amyloidosis associated with carpal tunnel syndrome, senile cardiac amyloidosis, familial amyloidotic polyneuropathy, and the amyloidosis associated with endocrine tumors. In certain embodiments, the diseases are Alzheimer's disease or type 2 diabetes.

Also provided are methods to inhibit or prevent α-synuclein/NAC fibril formation, methods to inhibit or prevent α-synuclein/NAC fibril growth, and methods to cause disassembly, disruption, and/or disaggregation of preformed α-synuclein/NAC fibrils and α-synuclein/NAC-associated protein deposits.

In certain embodiments, the synuclein diseases or synucleinopathies treated, prevented or whose symptoms are ameliorated by the compounds and compositions provided herein include, but are not limited to diseases associated with the formation, deposition, accumulation, or persistence of synuclein fibrils, including α-synuclein fibrils. In certain embodiments, such diseases include Parkinson's disease, familial Parkinson's disease, Lewy body disease, the Lewy body variant of Alzheimer's disease, dementia with Lewy bodies, multiple system atrophy, and the Parkinsonism-dementia complex of Guam.

### H. Methods of Use of the Compounds and Compositions for Detection

Methods for detecting the presence or absence of amyloid or α-synuclein aggregates in a biological sample are provided. These methods include contacting a biological sample with a selected compound, wherein the compound is labeled with a detectable substance, for example, with a radionucleotide, phosphorescent compound, fluorescent compound, fluorescent protein, paramagnetic compound, metal chelators, or enzyme, all of which are readily detectable in various assays and diagnostics know to those skilled in the art, and then detecting the detectable substance bound to the amyloid or α-synuclein aggregates in the biological sample.

Methods for imaging the presence or absence of amyloid or α-synuclein aggregates in the body or biological tissues are provided. These methods include contacting amyloid or α-synuclein aggregates in the body with a compound, wherein the compound is labeled with detectable substance, for example, with a radionucleotide, phosphorescent compound, fluorescent compound, fluorescent protein, paramagnetic compound, metal chelator, or enzyme, and detecting the detectable substance bound to the amyloid or α-synuclein aggregates in the body or biological tissues.

### Diagnostic Applications

Accordingly, the compounds can also be used as diagnostic agents to detect the presence or absence of amyloid or α-synuclein aggregates in a biological sample or in vivo in a subject. Furthermore, detection of amyloid or α-synuclein aggregates using the compounds can be used to diagnose amyloidosis or synucleopathies in a subject.

Compounds which interact with Aβ amyloid or α-synuclein or derivatives thereof are also disclosed herein. The compounds can be used for a number of important diagnostic and/or therapeutic applications as described herein.

In another embodiment, a compound is used in vivo to detect, and if desired, quantitate, Aβ amyloid or α-synuclein deposition in a subject, for example, to aid in the diagnosis of Aβ amyloidosis or synucleopathies in the subject. To aid in detection, the compound can be modified with a detectable substance, preferably ^{99m}Tc or radioactive iodine or fluorine. Methods for labeling peptide compounds with technetium are known in the art. A modifying group can be chosen that provides a site at which a chelation group for ^{99m}Tc can be introduced, such as a derivative of cholic acid, which has a free amino group. Also provided are compounds labeled with radioactive fluorine. For example the isotope of radioactive fluorine can be incorporated to create a diagnostic agent. Preferably, ¹⁸F for positron emission tomography (PET) or single photon emission computed tomography (SPECT) studies.

The following non-limiting Examples are given by way of illustration only and are not considered a limitation of the subject matter, many apparent variations of which are possible without departing from the spirit or scope thereof.

### EXAMPLES

### General Experimental Procedures

All solvents were distilled before use and were removed by rotary evaporation at temperatures up to 35° C. Merck silica gel 60, 200-400 mesh, 40-63 μm, was used for silica gel flash chromatography. TLC was carried out using Merck DC•plastikfolien Kieselgel 60 F₂₅₄, first visualised with a UV lamp, and then by dipping in a vanillin solution (1% vanillin, 1% H₂SO₄ in EtOH), and heating. Mass spectra were recorded on a Kratos MS-80 instrument. NMR spectra, at 25° C., were recorded at 500 or 300 MHz for ¹H and 125 or 75 MHz for ¹³C on Varian INOVA-500 or VXR-300 spectrometers. Chemical shifts are given in ppm on the δ scale referenced to the solvent peaks CHCl₃ at 7.25 and CDCl₃ at 77.0 ppm or (CH₃)₂CO at 2.15 and (CD₃)₂CO at 30.5 ppm or CH₃OD at 3.30 and CD₃OD at 39.0 ppm.

### HPLC Conditions

The analytical HPLC equipment consisted of a Waters 717 autosampler, 600 pump and controller, and a 2487 UV detector controlled by Omega software for method 2, and a Waters 717 autosampler, 600 pump and controller, and a 490 UV detector controlled by Millennium software for method 1. Samples were analysed by using an RP-18 semi-preparative column (Phenomenex Prodigy 5 mm C18 100A, 250×4.6 mm) with a guard column (Phonomenex SecurityGuard cartridge containing a C18 ODS 4×3 mm, 5 mm column) fitted at 30° C. Samples (5 mL) were analysed using a mobile phase flow rate of 5.0 mL/min, with UV detection at 280 nm.

### Solvent A—CH₃CN

Solvent B—H₂O containing 0.1% TFA

Method 1

| Time (minutes) | solvent A | solvent B |
|---|---|---|
| 0 | 11 | 89 |
| 20 | 11 | 89 |
| 30 | 100 | 0 |
| 31 | 11 | 89 |
| 40 | 11 | 89 |

HPLC Method 2 (for Compounds DC-0051-B1 through DC-0051-B4)

The method 2 constitutes using a C18 column with 2.1×50 mm dimensions. The run time is set at 7 minutes. The mobile phase included (A) acetonitrile with 0.05% TFA, and (B) distilled water with 0.05% TFA. All runs with method 2 employed a gradient elution from 10% to 90% of solvent A.

### Example 1

### HPLC Method

For the following synthetic methods this HPLC method was employed. Samples were analysed using an Agilent HP1100 instrument, operated with EzChrom Elite software, and fitted with a C18 column (Phenomenex Prodigy 5 μm 100A, 250×4.6 mm) with a guard column (Phenomenex ODS 4×3 mm, 5 μm) held at 30° C. Peaks were detected at 280 nm. The mobile phase was acetonitrile in water (with 0.1% TFA): t₀=11%, t₂₀=11%, t₃₀=100%, t₃₁=11%, t₄₀=11%. The flow rate was 1 mL/min and the injection volume of 5 μL.

Overview of Synthesis of N-(2-fluoro-4,5-dihydroxyphenyl)-3,4-dihydroxy benzamide (DC51-F5)

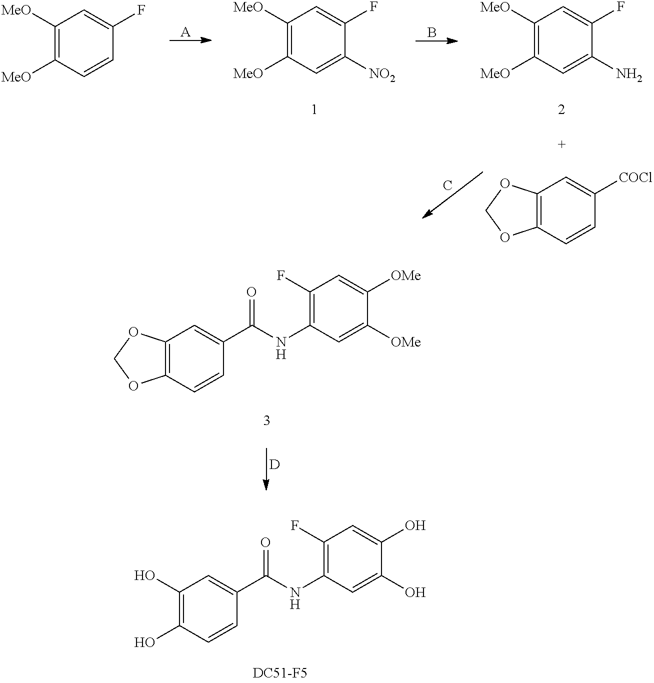

Nitration (I. M. Takakis et al. I Heterocyclic Chem. 1991, 28, 625-634) of commercially available 4-fluoroveratrole gave, as expected, just the 2-nitro isomer 1, as shown by NMR. Reduction using tin II chloride (A. Kamal et al. Bioorg. Med. Chem. 2007, 15 (22), 6868-6875) of this gave the aniline 2, which was immediately reacted with 3,4-methylenedioxybenzoyl chloride to give the anilide 3. Deprotection with boron tribromide under standard conditions gave the free phenolic amide, DC51-F5 (N-(2-fluoro-4,5-dihydroxyphenyl)-3,4-dihydroxybenzamide) in reasonable yield.

### Step A: Preparation of 1-fluoro-4,5-dimethoxy-2-nitrobenzene (1)

To ice cold nitric acid (10 ml) was added dropwise with stirring 4-fluoroveratrole (1 g). The mixture was stirred at 0° C. for 15 minutes, then brought to RT for 15 mins. The orange solution was poured over ice and the resultant off white solid was filtered, washed with water then dried to give 1-fluoro-4,5-dimethoxy-2-nitrobenzene (1) (1.25 g, 97%).

¹H NMR (CDCl₃, 500 MHz) 7.58 (1H, d, J 7 Hz, H3), 6.72 (1H, d, J 12 Hz, H6), 3.96 (3H, s) and 3.93 (3H, s).

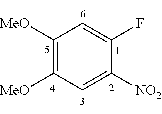

### Step B: Preparation of 2-fluoro-4,5-dimethoxyaniline (2)

A solution of 1-fluoro-4,5-dimethoxy-2-nitrobenzene (1) (0.28 g) in ethyl acetate/95% ethanol (7 ml/7 ml) and tin(II) chloride (1.0 g) was heated to 70° C. with stirring for 3 h, when more tin(II) chloride (1.0 g) was added and the mixture heated for a further 2 h, then left stirring at RT overnight. The solvents were removed in vacuo, then methanol/DCM (1:10) and sodium bicarbonate (sat.) were added. The mixture was filtered through ceelite, then the ceelite washed with further methanol/DCM (1:10). The organic phase was separated, dried, and evaporated in vacuo to give the product as a dark brown solid. This was used directly in the next step.

¹H NMR (CDCl₃, 300 MHz) 6.57 (1H, d, J 13 Hz, H3), 6.34 (1H, d, J 9 Hz, H6), 3.77 (3H, s) and 3.74 (3H, s).

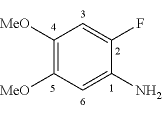

### Step C: Preparation of 3,4-Methylenedioxy-N-(4,5-dimethoxy-2-fluoro phenyl)benzamide (3)

A suspension of piperonylic acid (0.51 g, 3.1 mmol) in thionyl chloride (5 ml) was refluxed in the absence of moisture for 2 hours when a clear solution had been formed. The excess thionyl chloride was removed by evaporation in vacuo to leave the acid chloride as an off-white solid.

A solution of the acid chloride in dichloromethane (20 ml) was added to a solution of 2-fluoro-4,5-dimethoxyaniline (2) (0.40 g, 2.9 mmol) in dichloromethane (5 ml) and then pyridine (1 ml) was added. The mixture was refluxed together for 3 hours, then left at RT overnight. The resultant brown solution was diluted with dichloromethane (100 ml) and methanol (15 ml), washed with hydrochloric acid (1M, 50 ml) then dried and evaporated in vacuo to give the product as a brown solid. Purification by column chromatography over silica gel eluting with 10 to 20% ethyl acetate in DCM gave 3,4-Methylenedioxy-N-(4,5-dimethoxy-2-fluoro phenyl)benzamide (3) as a brown solid (480 mg, 57%).

M/z found 342.0767. C₁₆H₁₄FNNaO₅ requires 342.0748.

¹H NMR((CD₃)₂CO, 500 MHz) 9.01 (1H, bs, NH), 7.72 (1H, dd, J 2, 8 Hz, H6), 7.63 (1H, t, J 8 Hz, H6′), 7.59 (1H, d, J 2 Hz, H2), 7.06 (1H, d, J 8 Hz, H5), 6.97 (111, d, J 12 Hz, H3′), 6.22 (2H, s), 3.93 (3H, s) and 3.90 (3H, s).

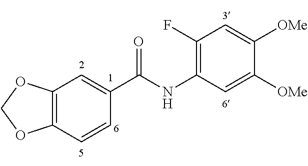

### Step D: Preparation of 3,4-Dihydroxy-N-(2-fluoro-4,5-dihydroxy phenyl)benzamide (DC51-F5)

To a solution of amide (3) (400 mg, 1.25 mmol) in dry dichloromethane (30 ml) at RT was added boron tribromide (1 ml). A pale green solution was first formed which rapidly formed an off-brown precipitate. This suspension was left at RT for 3 h, then methanol (then 20 ml) was added carefully and the resultant brown solution left at RT overnight, when a yellow solid was formed.

The solvents were removed in vacuo to leave about 1 ml, then methanol (dropwise then 30 ml) was added carefully. This was repeated 4 times, then solvents removed in vacuo to leave the product as an off-white solid. The solid was dissolved in a minimum amount of methanol, then dichloromethane added until just cloudy. The mixture was left in the fridge overnight when the product was formed as an off white crystalline solid (261 mg, 78%).

Hplc 18.12 minutes.

M]z found 278.0479. C₁₃H₉FNO₅ requires 278.0470.

¹H NMR (CD₃OD, 300 MHz) 7.36 (1H, d, J 2 Hz, H2), 7.32 (1H, dd, J 2, 9 Hz, H6), 7.02 (1H, d, J 8 Hz, H6′), 6.83 (1H, d, J 9 Hz, H5) and 6.60 (1H, d, J 12 Hz, H3′).

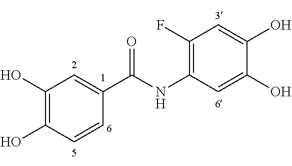

### Example 2

### Overview of Synthesis of Positional isomers N-(3-fluoro-4,5-dihydroxyphenyl)-3,4-dihydroxy benzamide (DC51-F4) and N-(2-fluoro-3,4-dihydroxyphenyl)-3,4-dihydroxy benzamide (DC51-F6)

3-Fluoroveratrole 4 was prepared by methylation⁴ of the commercially available 3-fluorocatechol. Nitration of 3-fluoroveratrole gave a mixture of the two isomeric products 5 and 6 in a 2:1 ratio (structures and ratio determined by NMR spectroscopy). Separation of these, followed by reduction with tin II chloride³ gave the anilines 7 and 8. Reaction of the anilines with 3,4-methylenedioxybenzoyl chloride gave the anilides 9 and 10. Deprotection with boron tribromide under standard conditions gave the free phenolic anilides, N-(3-fluoro-4,5-dihydroxyphenyl)-3,4-dihydroxybenzamide (DC51-F4) and N-(2-fluoro-3,4-dihydroxyphenyl)-3,4-dihydroxybenzamide (DC51-F6) in reasonable yield.

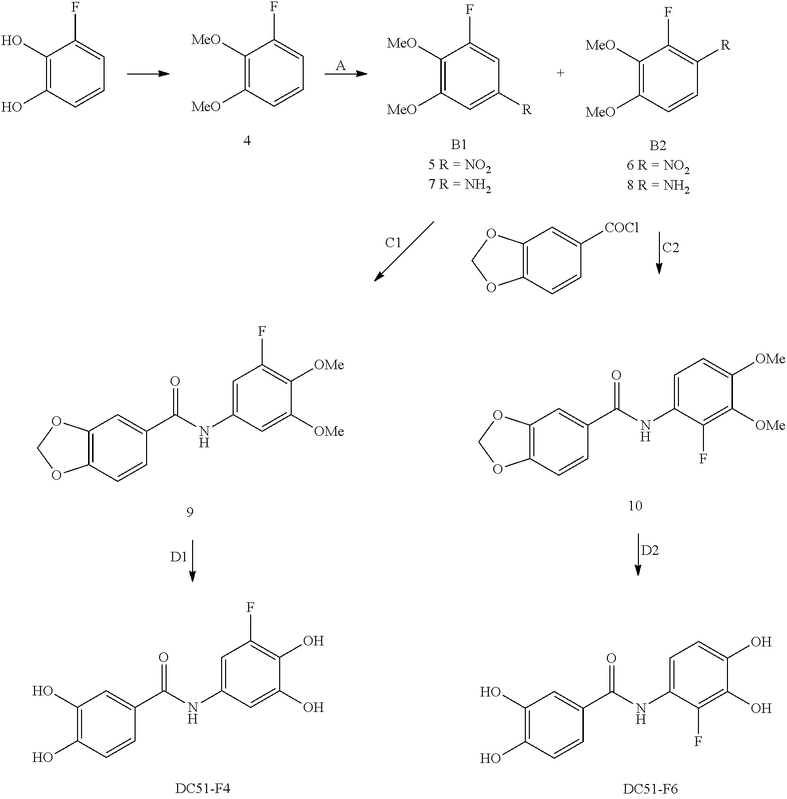

### Step A: Preparation of 1-fluoro-2,3-dimethoxy-5-nitrobenzene (5) and 1-fluoro-2,3-dimethoxy-6-nitrobenzene (6)

To ice cold nitric acid (10 ml) was added dropwise with stirring 3-fluoroveratrole (2 g). The mixture was stirred at 0° C. for 15 minutes, then brought to RT for 15 mins. The orange solution was poured over ice and the resultant off white solid was filtered, washed with water then dried.

Column chromatography over silica eluting with pet ether/DCM (1:1) gave the pure 1-fluoro-2,3-dimethoxy-5-nitrobenzene (5) (V M. Cervera et al. Tetrahedron 1996, 52 (7), 2557-2564) (1.20 mg, 50%) ¹HNMR (CDCl₃, 500 MHz) 7.68 (1H, dd, J 2, 11 Hz, H6), 7.61 (1H, dd, J 1, 2 Hz, H4), 4.06 (3H, d, 2 Hz) and 3.96 (3H, s).

This first fraction was followed by pure 1-fluoro-2,3-dimethoxy-6-nitrobenzene (6) (0.550 mg, 23%).

¹H NMR (CDCl₃, 500 MHz) 7.86 (1H, dd, J 9, 10 Hz, H5), 6.75 (1H, dd, J 2, 9 Hz, H4), 3.96 (3H, s) and 3.97 (3H, d, 2 Hz).

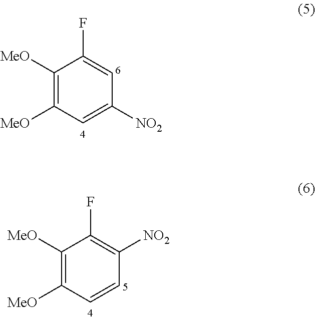

### Step B1: Preparation of 3-fluoro-4,5-dimethoxyaniline (7)

A solution of 1-fluoro-2,3-dimethoxy-5-nitrobenzene (5) (0.56 g) in ethyl acetate/95% ethanol (14 ml/14 ml) and tin(II) chloride (2.0 g) was heated to 70° C. with stirring for 3 h, when more tin(II) chloride (1.0 g) was added and the mixture heated for a further 2 h, then left stirring at RT overnight. The solvents were removed in vacuo, then methanol/DCM (1:10) and sodium bicarbonate (sat.) were added. The mixture was filtered through ceelite, then the ceelite washed with further methanol/DCM (1:10). The organic phase was separated, dried, and evaporated in vacuo to give aniline (7) as a dark brown solid. This was used directly in the following reaction.

### Step B2: Preparation of -fluoro-3,4-dimethoxy-aniline (8)

A solution of 1-fluoro-2,3-dimethoxy-6-nitrobenzene (6) (0.56 g) in ethyl acetate/95% ethanol (14 ml/14 ml) and tin(II) chloride (2.0 g) was heated to 70° C. with stirring for 3 h, when more tin(II) chloride (1.0 g) was added and the mixture heated for a further 2 h, then left stirring at RT overnight. The solvents were removed in vacuo, then methanol/DCM (1:10) and sodium bicarbonate (sat.) were added. The mixture was filtered through ceelite, then the ceelite washed with further methanol/DCM (1:10). The organic phase was separated, dried, and evaporated in vacuo to give aniline (8) as a dark brown solid. This was used directly in the following reaction.

### Step C1: Preparation of 3,4-Methylenedioxy-N-(4,5-dimethoxy-3-fluoro phenyl)benzamide (9)

A suspension of piperonylic acid (0.57 g, 3.45 mmol) in thionyl chloride (5 ml) was refluxed in the absence of moisture for 2 hours when a clear solution had been formed. The excess thionyl chloride was removed by evaporation in vacuo to leave the acid chloride as an off-white solid.

A solution of the acid chloride in dichloromethane (20 ml) was added to a solution of 3-fluoro-4,5-dimethoxy-aniline (7) (0.45 g, 3.3 mmol) in dichloromethane (5 ml) and then pyridine (1 ml) was added. The mixture was refluxed together for 3 hours, then left at RT overnight. The resultant brown solution was diluted with dichloromethane (100 ml) and methanol (15 ml), washed with hydrochloric acid (1M, 50 ml) then dried and evaporated in vacuo to give the product as a brown solid. Purification by column chromatography over silica gel eluting with 10 to 20% ethyl acetate in DCM gave amide (9) as an off-white solid (400 mg, 43%).

M/z found 342.0751. C₁₆H₁₄FNNaO₅ requires 342.0748.

¹H NMR (CDCl₃, 300 MHz) 7.58 (1H, bs, NH), 7.36 (1H, dd, J 2, 9 Hz, H6), 7.33 (1H, d, J 2 Hz, H2), 7.18 (1H, t, J 2 Hz, H6′), 6.93 (1H, dd, J 2, 13 Hz, H2′), 6.87 (1H, d, J 9 Hz, H5), 6.06 (2H, s), 3.90 (3H, s) and 3.89 (3H, s).

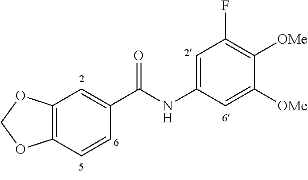

### Step C2: Preparation of 3,4-Methylenedioxy-N-(3,4-dimethoxy-2-fluorophenyl)benzamide (10)

A suspension of piperonylic acid (0.57 g, 3.45 mmol) in thionyl chloride (5 ml) was refluxed in the absence of moisture for 2 hours when a clear solution had been formed. The excess thionyl chloride was removed by evaporation in vacuo to leave the acid chloride as an off-white solid.

A solution of the acid chloride in dichloromethane (20 ml) was added to a solution of 2-fluoro-3,4-dimethoxy-aniline (8) (0.45 g, 3.3 mmol) in dichloromethane (5 ml) and then pyridine (1 ml) was added. The mixture was refluxed together for 3 hours, then left at RT overnight. The resultant brown solution was diluted with dichloromethane (100 ml) and methanol (15 ml), washed with hydrochloric acid (1M, 50 ml) then dried and evaporated in vacuo to give the product as a brown solid. Purification by column chromatography over silica gel eluting with 10 to 20% ethyl acetate in DCM gave amide (10) as an off-white solid (430 mg, 46%).

¹H NMR (CDCl₃, 300 MHz) 7.97 (1H, t, J 9 Hz, H6′), 7.71 (1H, bs, NH), 7.40 (1H, bd, J 9 Hz, H6), 7.36 (1H, bs, H2), 6.88 (1H, d, J 9 Hz, H5), 6.70 (1H, bd, J 10 Hz, H5′), 6.06 (2H, s), 3.94 (3H, s) and 3.87 (3H, s).

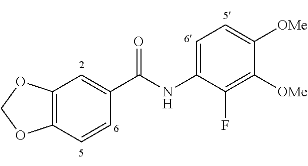

### Step D1: Preparation of 3,4-Dihydroxy-N-(3-fluoro-4,5-dihydroxyphenyl)benzamide (DC51-F4)

To a solution of amide (9) (400 mg, 1.25 mmol) in dry dichloromethane (30 ml) at RT was added boron tribromide (1 ml). A pale green solution was first formed with a light white precipitate. This suspension was left at R. for 3 h, then methanol (dropwise then 20 ml) was added carefully and the resultant brown solution left at RT overnight, when a yellow solid was formed.

The solvents were removed in vacuo to leave about 1 ml, then methanol (dropwise then 30 ml) was added carefully. This was repeated 4 times, then solvents removed in vacuo to leave the product as an off-white solid. The solid was dissolved in a minimum amount of methanol, then dichloromethane added until just cloudy. The mixture was left in the fridge overnight when (DC51-F4) was formed as an off white crystalline solid (271 mg, 81%).

HPLC 22.44 minutes.

M/z found 278.0455. C₁₃H₉FNO₅ requires 278.0470.

¹H NMR (CD₃OD, 300 MHz) 7.34 (1H, d, J 2 Hz, H2), 7.29 (1H, dd, J 2, 9 Hz, H6), 6.97 (1H, bs, H6′), 6.96 (1H, dd, J 2, 11 Hz, H2′) and 6.82 (1H, d, J 9 Hz, H5).

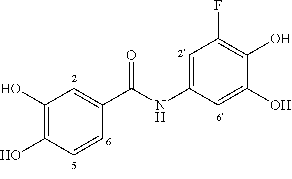

### Step D2: Preparation of 3,4-Dihydroxy-N-(2-fluoro-3,4-dihydroxy phenyl)benzamide (DC51-F6)

To a solution of amide (10) (430 mg, 1.34 mmol) in dry dichloromethane (30 ml) at RT was added boron tribromide (1 ml). A pale orange solution was first formed with a light white ppt. This suspension was left at RT for 3 h, then methanol (dropwise then 20 ml) was added carefully and the resultant brown solution left at RT overnight, when a yellow solid was formed.

The solvents were removed in vacuo to leave about 1 ml, then methanol (dropwise then 30 ml) was added carefully. This was repeated 4 times, then solvents removed in vacuo to leave the product as an off-white solid. The solid was dissolved in a minimum amount of methanol, then dichloromethane added until just cloudy. The mixture was left in the fridge overnight when the product was formed as a brown crystalline solid (241 mg, 67%).

HPLC 12.13 minutes.

M/z found 278.0490. C₁₃H₉FNO₅ requires 278.0470.

1H NMR (CD₃OD, 300 MHz) 7.38 (1H, d, J 2.5 Hz, H2), 7.33 (1H, dd, J 2.5, 9 Hz, H6), 6.83 (1H, d, J 9 Hz, H5), 6.81 (1H, t, J 9 Hz, H6′) and 6.58 (1H, dd, J 2.5, 9 Hz, H5′).

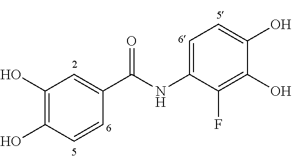

### Example 3

### Overview of Synthesis of N-(3,4-dihydroxyphenyl)-2-fluoro-4,5-dihydroxy benzamide (DC51-F2)

Oxidation of commercially available 6-fluoroveratraldehyde with ‘Jones reagent'⁶ gave the acid 11 in good yield. Formation of’ the acid chloride from this and condensation with 3,4-methylenedioxyaniline then gave the anilide 12. Deprotection with boron tribromide under standard conditions gave the free phenolic anilide, DC51-F2 in good yield.

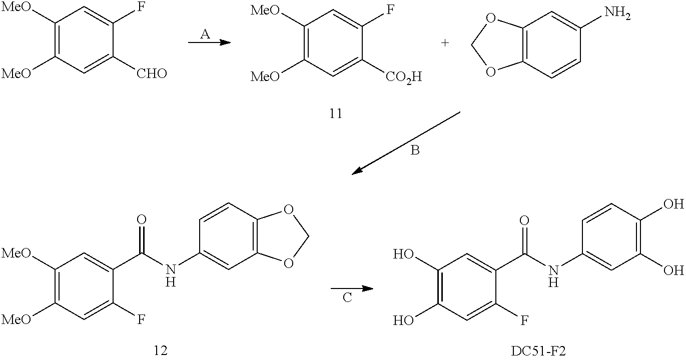

### Step A: Preparation of 2-fluoro-4,5-dimethoxybenzoic acid (11)

To a stirred solution of 2-fluoro-4,5-dimethoxybenzaldehyde (0.46 g, 2.5 mmol) in acetone (20 ml) was added Jones reagent (6 ml) dropwise and the mixture stirred at RT for 4 h (P. B. Wakchaure et al. Tetrahedron 2008, 64, 1786-1791.). The mixture was diluted with water, extracted into ethyl acetate then dried and evaporated in vacuo to give the crude acid. Recrystallisation from ethyl acetate/pet ether (40-60) gave the pure acid as a brown crystalline solid (H. B. Stegmann et al. J.C.S. Perkin Trans 2, 1994, 547-555).

¹H NMR (CDCl₃, 300 MHz) 7.43 (1H, d, J 7 Hz, H6), 6.66 (1H, d, 13 Hz, H3), 3.93 (3H, s) and 3.90 (3H, s).

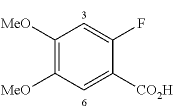

### Step B: Preparation of 4,5-Dimethoxy-2-fluoro-N-(3,4-methylene dioxy phenyl)benzamide (12)

A suspension of 2-fluoro-4,5-dimethoxybenzoic acid (11) (0.40 g, 2.0 mmol) in thionyl chloride (5 ml) was refluxed in the absence of moisture for 1 hour when a clear solution had been formed. The excess thionyl chloride was removed by evaporation in vacuo to leave the acid chloride as an off-white solid.

A solution of the acid chloride in dichloromethane (15 ml) was added to a solution of 3,4-methylenedioxyaniline (0.33 g, 2.4 mmol) in dichloromethane (5 ml) and then pyridine (0.5 ml) was added. The mixture was refluxed together for 2 hours, then left at RT overnight. The resultant brown solution was diluted with DCM I MeOH (5:1)(50 ml) washed with hydrochloric acid (1M, 25 ml) then dried and evaporated in vacuo to give the product as a brown solid. The product was columned over silica gel when 10 to 20% ethyl acetate in DCM gave amide (12) as an off-white solid (550 mg, 86%).

M/z found 342.0759. C₁₆H₁₄FNNaO₅ requires 342.0748.

¹H NMR((CD₃)₂CO, 300 MHz) 9.13 (1H, bs, NH), 7.62 (1H, bs, H2′), 7.48 (1H, d, J 8 Hz, H6), 7.24 (1H, bd, J 8 Hz, H6′), 7.01 (1H, d, J 13 Hz, H3), 6.92 (1H, d, J 9 Hz, H5′), 6.10 (2H, s), 4.01 (3H, s) and 3.96 (3H, s).

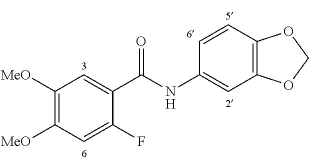

### Step C: Preparation of 4,5-Dihydroxy-2-fluoro-N-(3,4-dihydroxy phenyl)benzamide (DC51-F2)

To a solution of amide (12) (500 mg, 1.6 mmol) in dry dichloromethane (30 ml) at RT was added boron tribromide (1 ml). A pale orange solution was first formed with a light white ppt. This suspension was left at RT for 3 h, then methanol (dropwise then 20 ml) was added carefully and the resultant brown solution left at RT overnight, when a yellow solid was formed.

The solvents were removed in vacuo to leave about 1 ml, then methanol (dropwise then 30 ml) was added carefully. This was repeated 4 times, and then solvents removed in vacuo to leave the product as an off-white solid. The solid was dissolved in a minimum amount of methanol, and then dichloromethane added until just cloudy. The mixture was left in the fridge overnight when (DC51-F2) was formed as an off white crystalline solid (320 mg, 73%).

HPLC 25.71 minutes.

M/z found 278.0488. C₁₃H₉FNO₅ requires 278.0470.

¹HNMR (CD₃OD, 300 MHz) 7.17-7.19 (2H, m, H6, H2′), 6.84 (1H, dd, J 2.5, 9 Hz, H6′), 6.72 (1H, d, J 9 Hz, H5′) and 6.60 (1H, d, J 13 Hz, H3).

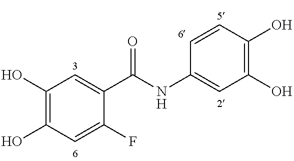

### Example 4

### Overview of Synthesis of N-(3,4-dihydroxyphenyl)-2-fluoro-3,4-dihydroxy benzamide (DC51-F1)

Aldehyde 13 could be prepared, as described previously by Ladd et al. using a reaction of hexamethylenetetramine and trifluoroacetic acid (D. L. Ladd et al. J. Org. Chem. 1981, 46 (1), 203-206). Oxidation of aldehyde 13 gave the benzoic acid 14, then formation of the acid chloride from this and condensation with 3,4-methylenedioxyaniline gave the anilide 15. Deprotection with boron tribromide under standard conditions gave the free phenolic anilide, DC51-F1 in good yield.

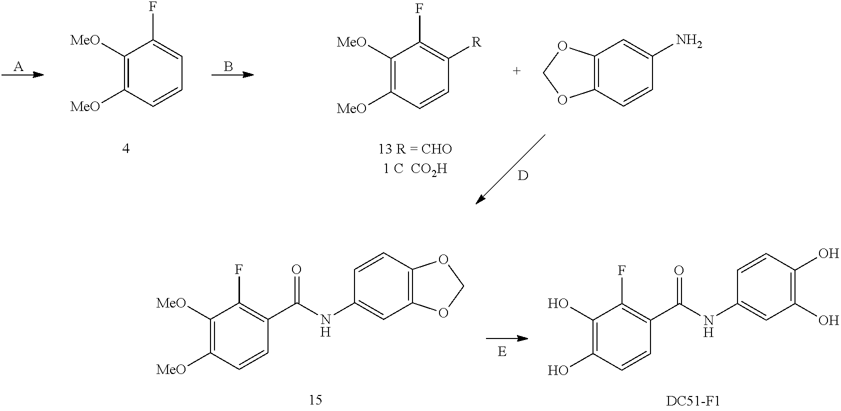

### Step A: Preparation of 1-Fluoro-2,3-dimethoxybenzene (4)

A suspension of 3-fluoro-catechol (3.7 g, 29 mmol) in acetone (30 ml) with potassium carbonate (12 g, 87 mmol) and methyl iodide (10 ml, 160 mmol) was stirred at RT for 24 h. The mixture was diluted with ethyl acetate, filtered, washed with water, dried and evaporated in vacuo to give the product as a pale yellow liquid (3.42, 76%).

¹H NMR (CDCl₃, 300 MHz) 7.00-6.90 (1H, m), 6.76-6.64 (1H, m), 3.91 (3H, d, 1 Hz) and 3.86 (3H, s).

### Step B: Preparation of 2-fluoro-3,4-dimethoxybenzaldehyde (13)

To a heated solution of hexamethylenetetraamine (2.2 g, 16 mmol) in TFA (10 ml) at 80° C. was added dropwise 3-fluoroveratrole (1.20 g, 7.8 mmol) in TFA (10 ml). The mixture was heated for an additional hour, then concentrated in vacuo.

Ice/water (30 ml) was added until the mixture turned cloudy, the mixture was stirred for 15 mins then made basic with solid sodium carbonate, stirred for 20 mins then extracted into ethyl acetate, washed with water dried and evaporated in vacuo.

Purification by column chromatography over silica gel eluting with ethyl acetate (0-50%) in dichloromethane gave aldehyde (13) (D. L. Ladd et al. Syn. Comm, 1985, 15 (1), 61-69) as a colourless gum (0.96 g, 68%).

¹H NMR (CDCl₃, 300 MHz) 10.19 (1H, s, CHO), 7.58 (1H, dd, J 7, 9 Hz, H6), 6.79 (1H, d, 9 Hz, H5) and 3.95 (6H, s).

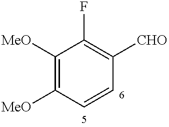

### Step C: Preparation of 2-Fluoro-3,4-dimethoxybenzoic acid (14)

To a stirred solution of 2-fluoro-3,4-dimethoxybenzaldehyde (13) (0.46 g, 2.5 mmol) in acetone (20 ml) was added Jones reagent (6 ml) dropwise and the mixture stirred at RT for 4 h.⁶ The mixture was diluted with water, extracted into ethyl acetate then dried and evaporated in vacuo to give the crude acid. Recrystallisation from ethyl acetate/pet ether (40-60) gave the pure acid (14) as a brown crystalline solid (H. B. Stegmann et al. J.C.S. Perkin Trans 2, 1994, 547-555).

¹H NMR((CD₃)₂CO₃ 300 MHz) 7.80 (1H, dd, J 7, 8 Hz, H6), 7.07 (1H, d, 8 Hz, H5), 4.06 (3H, s) and 3.95 (3H, s).

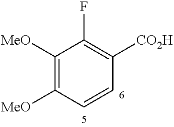

### Step D: Preparation of 3,4-Dimethoxy-2-fluoro-N-(3,4-methylene dioxyphenyl)benzamide (15)

A suspension of 2-fluoro-3,4-dimethoxybenzoic acid (14) (0.50 g, 2.5 mmol) in thionyl chloride (5 ml) was refluxed in the absence of moisture for 1 hour when a clear solution had been formed. The excess thionyl chloride was removed by evaporation in vacuo to leave the acid chloride as an off-white solid.

A solution of the acid chloride in dichloromethane (15 ml) was added to a solution of 3,4-methylenedioxyaniline (0.41 g, 3.0 mmol) in dichloromethane (5 ml) and then pyridine (0.5 ml) was added. The mixture was refluxed together for 2 hours, then left at RT overnight. The resultant brown solution was diluted with DCM/MeOH (5:1)(50 ml) washed with hydrochloric acid (1M, 25 ml) then dried and evaporated in vacuo to give the product as a brown solid. The product was columned over silica gel when 10 to 20% ethyl acetate in DCM gave amide (15) as an off-white solid (630 mg, 79%). M/z found 342.0760. C₁₆H₁₄FNNaO₅ requires 342.0748.

¹H NMR((CD₃)₂CO₃ 300 MHz) 9.22 (1H, bs, NH), 7.66-7.60 (2H, m, H2′, H6), 7.25 (1H, bd, J 8 Hz, H6′), 7.09 (1H, bd, J 9 Hz, H5), 6.92 (1H, d, J 9 Hz, H5′), 6.09 (2H, s), 4.01 (3H, s) and 3.96 (3H, s).

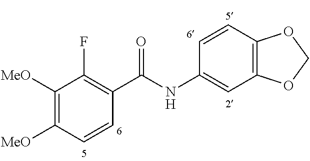

### Step E: Preparation of 3,4-Dihydroxy-2-fluoro-N-(3,4-dihydroxyphenyl)benzamide (DC51-F1)

To a solution of amide (15) (500 mg, 1.6 mmol) in dry dichloromethane (30 ml) at r.t. was added boron tribromide (1 ml). A pale orange solution was first formed with a light white ppt. This suspension was left at RT for 3 h, then methanol (dropwise then 20 ml) was added carefully and the resultant brown solution left at RT overnight, when a yellow solid was formed.

The solvents were removed in vacuo to leave about 1 ml, then methanol

(dropwise then 30 ml) was added carefully. This was repeated 4 times, then solvents removed in vacuo to leave the product as an off-white solid. The solid was dissolved in a minimum amount of methanol, then dichloromethane added until just cloudy. The mixture was left in the fridge overnight when (DC51-F1) was formed as a brown crystalline solid (283 mg, 65%).

HPLC 15.10 minutes.

M/z found 278.0488. C₁₃H₉FNO₅ requires 278.0470.

¹H NMR (CD₃OD, 300 MHz) 7.21 (1H, d, J 2.5, H2′), 7.09 (1H, t, J 9 Hz, H6), 6.85 (1H, dd, J 2.5, 9 Hz, H6′), 6.72 (1H, d, J 9 Hz, H5′) and 6.66 (1H, dd, J 1, 9 Hz, H5).

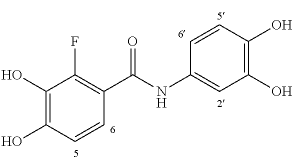

### Example 5

### Overview of Synthesis of N-(3,4-dihydroxyphenyl)-3-fluoro-4,5-dihydroxy benzamide (DC51-F3)

Work by Clark (M. T. Clark and D. D. M.iller. J. Org. Chem. 1986, 51, 4072-4073) showed that the required formyl product can be prepared in a three step process from 2-fluoro-6-methoxyphenol, which in turn can be prepared from commercially available 3-fluoroanisole. Within the timeframe of the work, 3-fluoroanisole was unavailable, so we looked for an alternative route to a 2-fluoro-6-alkoxyphenol.

Alkylation of 3-fluorocatechol with one equivalent of benzyl bromide (benzyl chosen as a bulkier group than methyl which should give a greater proportion of the monoalkylated products) gave a 2:1 mixture of mono and di-benzylated products. Separation of these gave the two mono-benzylated products 16b and 16c as an inseparable mixture. Treatment of this mixture under the formylation conditions used for 2-fluoro-6-methoxyphenol (M. T. Clark and D. D. M.iller. J. Org. Chem. 1986, 51, 4072-4073) gave two readily separated aldehydes 17b and 17c. Comparison of their NMR spectra with previously formed compounds showed them to be the two expected products. Methylation⁴ of the phenol gave the methoxide 18 which upon oxidation then gave the acid 19. Formation of the acid chloride from this and condensation with 3,4-methylenedioxyaniline gave the anilide 20. Deprotection with boron tribromide under standard conditions gave the free phenolic anilide, DC51-F3 although in poor overall yield.

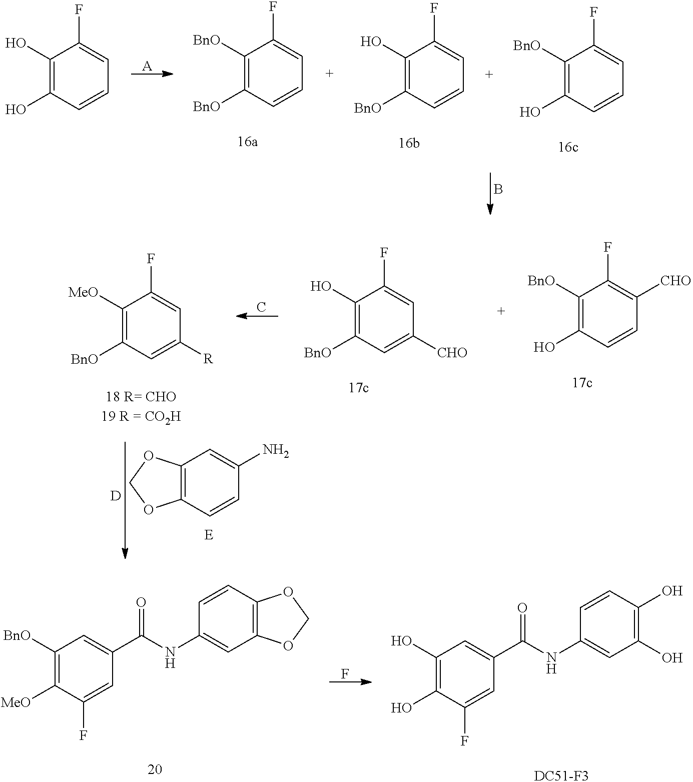

### Step A: Preparation of 2,3-Dibenzyloxy-1-fluorobenzene, 6-benzyloxy-2-fluorophenol and 2-benzyloxy-3-fluorophenol (16a) (16b) and (16c)

A mixture of 3-fluorocatachol (2.0 g, 16 mmol), benzyl bromide (2.67 g, 15.6 mmol) sodium iodide (1.2 g, 7.8 mmol) and potassium carbonate (6 g, 47 mmol) in acetonitrile (100 ml) was sonicated for 5 minutes, then stirred at RT overnight. Water (100 ml) was added, then the solution made acidic with 3M HCl and extracted with ethyl acetate, washed with brine, dried and evaporated in vacuo to give an orange gum. Separation by column chromatography over silica gel eluting with DCM/Pet ether (1:1) gave 2,3-Dibenzyloxy-1-fluorobenzene (16a) as a colourless oil (1.2 g, 25%) followed by a mixture of 6-benzyloxy-2-fluorophenol (16b) and 2-benzyloxy-3-fluorophenol (16c) as a colourless oil (2.05 g, 60%).

¹H NMR (CDCl₃, 300 MHz) 7.42-7.35 (5H, m, Bn), 6.93-6.84 (1H, m), 6.73-6.60 (2H, m), 5.57 (1H, s, OH), 5.14 (1H, s) and 5.13 (1H, s).

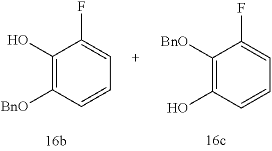

### Step B: Preparation of 6-Benzyloxy-2-fluorophenol (17b)

The mixture of phenols (16b) and (16c) (2.0 g, 9.2 mmol) was added to a solution of 33% dimethylamine (2.5 g, 18 mmol) and 37% formaldehyde (1.5 ml. 18 mmol) in absolute ethanol (15 ml). The mixture was heated at reflux for 2 h, cooled and evaporated to give a solid. Iodomethane (18 ml, 145 mmol) was added to a solution of this solid in chloroform (60 ml) and the mixture stirred for 18 h at RT. The white solid was filtered off then heated to 120° C. in acetic acid (6 ml) and water (6 ml), when HMTA (1.5 g, 11 mmol) was added to the mixture. The mixture was stirred at 120° C. for 2 h then c.HCl (0.25 ml) was added and heating continued for 5 mins. The cooled mixture was extracted into ether, dried and evaporated in vacuo. Purification by column chromatography over silica gel eluting with 0-100% DCM in pet ether (40-60) gave aldehyde (17b) as a white solid (0.25 g, 11%).

¹H NMR (CDCl₃, 300 MHz) 9.76 (1H, d, J 1 Hz, CHO), 7.45-7.37 (5H, m, Bn), 7.33 (1H, t, J 2 Hz, H6), 7.29 (1H, dd, J 2, 11 Hz, H2) and 5.19 (2H, s).

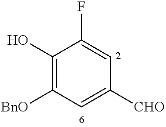

### Step C: Preparation of 5-Benzyloxy-3-fluoro-4-methoxybenzaldehyde⁹ (18)

A suspension of phenol (17b) (250 mg, 29 mmol) in acetone (10 ml) with potassium carbonate (1 g, 7 mmol) and methyl iodide (2 g, 14 mmol) was stirred at RT for 24 h. The mixture was diluted with ethyl acetate, filtered, washed with water, dried and evaporated in vacuo to give the product as a pale yellow liquid (250 mg, 95%).

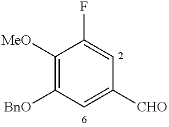

### Step D: Preparation of 5-Benzyloxy-3-fluoro-4-methoxybenzoic acid (19)

To a stirred solution of aldehyde (18) (0.25 g, 1.02 mmol) in acetone (20 ml) was added Jones reagent (6 ml) dropwise.⁶ The mixture was stirred at RT for 4 h then diluted with water, extracted into ethyl acetate then dried and evaporated in vacuo to give the crude acid (250 mg, 94%).

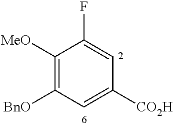

Step E: Preparation of 5-Benzyloxy-3-fluoro-4-methoxy-N-(3,4-methylene dioxy phenyl)benzamide (20)

A suspension of 5-benzyloxy-3-fluoro-4-methoxybenzoic (19) (0.25 g, 0.9 mmol) in thionyl chloride (5 ml) was refluxed in the absence of moisture for 1 hour when a clear solution had been formed. The excess thionyl chloride was removed by evaporation in vacuo to leave the acid chloride as an off-white solid.

A solution of the acid chloride in dichloromethane (15 ml) was added to a solution of 3,4-methylenedioxyaniline (0.15 g, 1.1 mmol) in dichloromethane (5 ml) and then pyridine (0.5 ml) was added. The mixture was refluxed together for 2 hours, then left at RT overnight. The resultant brown solution was diluted with DCM/MeOH (5:1)(50 ml) washed with hydrochloric acid (1M, 25 ml) then dried to give the product as a brown solid. The product was columned over silica gel when 10 to 20% ethyl acetate in DCM gave amide (20) as an off-white solid (320 mg, 89%).

¹H NMR (CDCl₃, 300 MHz) 7.48-7.33 (5H, m, Bn), 7.32-7.29 (2H, m, H6, H2′), 7.16 (1H, dd, J 2, 12 Hz, H2), 6.85 (1H, dd, J 2, 9 Hz, H6′), 6.77 (1H, d, J 9 Hz, H5′), 5.97 (2H, s), 5.19 (21-1, s) and 4.00 (31-1, s).

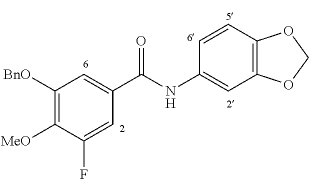

### Step F: Preparation of 4,5-Dihydroxy-3-fluoro-N-(3,4-dihydroxyphenyl)benzamide (DC51-F3)

To a solution of amide (20) (340 mg, 0.86 mmol) in dry dichloromethane (30 ml) at RT was added boron tribromide (1 ml). A pale orange solution was first formed with a light white ppt. This suspension was left at RT for 3 h, then methanol (dropwise then 20 ml) was added carefully and the resultant brown solution left at RT overnight, when a yellow solid was formed.

The solvents were removed in vacuo to leave about 1 ml, then methanol (dropwise then 30 ml) was added carefully. This was repeated 4 times, then solvents removed in vacuo to leave the product as a brown solid. The solid was freeze dried to give (DC51-F3) as a brown solid (229 mg, 95%).

HPLC 20.58 minutes.

M/z found 280.0600. C₁₃H₁₁FNO₅ requires 280.0616.

¹H NMR (CD₃OD, 300 MHz) 7.21 (1H, dd, J 2.5, 12 Hz, H2), 7.20 (1H, bs, H2′), 7.16 (1H, d, J 2.5 Hz, H6), 6.85 (1H, dd, J 2.5, 9 Hz, H6′) and 6.72 (1H, d, J 9 Hz, H5′).

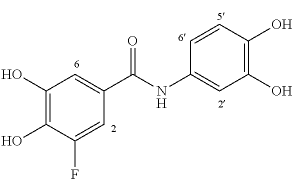

Two alternative preparations of DC51-F3 could start either from 3-fluoroanisole, to form 3-fluoro-4,5-dimethoxybenzaldehyde or (as shown below) introduce the fluoride onto the benzene ring at a later stage. One possibility would be the use of select fluor on compound (21) where there is just one free ortho or para position. After methylation the nitrile group could be hydrolysed to the required acid.

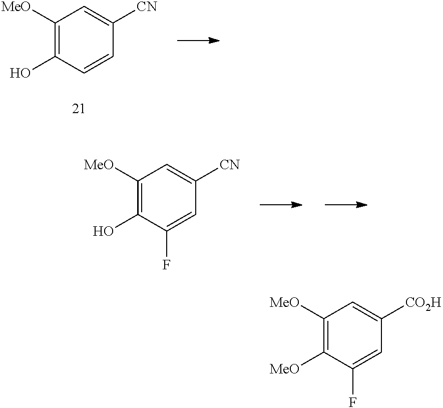

### Example 6

### Compounds Provided Herein are Potent Disrupters of Alzheimer's Aβ 1-42 Fibrils

The compounds prepared in the preceding Examples were found to be potent disrupters/inhibitors of Alzheimer's disease Aβ fibrils. In a set of studies, the efficacy of certain compounds provided herein to cause a disassembly/disruption of pre-formed amyloid fibrils of Alzheimer's disease (i.e. consisting of Aβ 1-42 fibrils) was analyzed.

### Part A—Thioflavin T Fluorometry Data

In one study, Thioflavin T fluorometry was used to determine the effects of the compounds, and of EDTA (as a negative control). In this assay Thioflavin T binds specifically to fibrillar amyloid, and this binding produces a fluorescence enhancement at 485 nm that is directly proportional to the amount of amyloid fibrils formed. The higher the fluorescence, the greater the amount of amyloid fibrils formed (Naki et al., Lab. Invest. 65:104-110, 1991; Levine III, Protein Sci. 2:404-410, 1993; Amyloid: Int. J. Exp. Clin. Invest. 2:1-6, 1995).

In this study, 25 μM of pre-fibrillized Aβ 1-42 (Bachem Inc) was incubated at 37° C. for 3 days, either alone, or in the presence of one of the compounds or EDTA (at Aβ:test compound weight ratios of 1:1, 1:0.1, 1:0.01 or 1:0.001). Following 3 days of co-incubation, 50 μl of each incubation mixture was transferred into a 96-well microtiter plate containing 150 μl of distilled water and 50 μl of a Thioflavin T solution (i.e. 500 mM Thioflavin T in 250 mM phosphate buffer, pH 6.8). The fluorescence was read at 485 nm (444 nm excitation wavelength) using an ELISA plate fluorometer after subtraction with buffer alone or compound alone, as blank).

The results of the 3-day incubations are presented below. For example, whereas EDTA caused no significant inhibition/disruption of Aβ 1-42 fibrils at all concentrations tested, the compounds (DC-0051, DC-0051-S1, S3, S4, S5, S6, S7, S8 and S9) all caused a dose-dependent disruption/disassembly of preformed Aβ 1-42 fibrils to some extent (Table 1). For example, compound DC-0051-S8 caused a significant (p<0.01) 87.9 +/−0.78% inhibition when used at an Aβ:test compound wt/wt ratio of 1:0.1, and a significant 56.0 +/−11.32% inhibition when used at an Aβ:test compound wt/wt ratio of 1:0.01 (Table 1). Under the same conditions (i.e. Aβ:test compound wt/wt ratio of 1:0.01), compound DC-0051 caused an 89.5+/−3.26% disruption, compound DC-0051-S5 caused an 80.0+/−0.63% disruption, and compound DC-0051-S9 caused an 84.1+/−4.28% disruption. This study indicated that the compound provided herein are disrupters/inhibitors of Alzheimer's disease type Aβ fibrils, and usually exert their effects in a dose-dependent manner.

TABLE 1
Thioflavin T Fluorometry Data-Disruption of Aβ 1-42 Fibrils by Test
Compounds (% inhibition of Aβ; for Aβ:test compound at given wt/wt ratio)

| Test Compound # | 1:1 (wt/wt) | 1:0.1 (wt/wt) | 1:0.01 (wt/wt) | 1:0.001 (wt/wt) |
|---|---|---|---|---|
| EDTA (control) | 0.0 ± 3.59% | 0.0 ± 4.41% | 0.2 ± 3.03% | 0.0 ± 1.54% |
| DC-0051 | 98.7 ± 0.07% | 89.5 ± 3.26% | 32.0 ± 4.31% | 10.9 ± 2.24% |
| DC0051-S1 | 96.4 ± 0.58% | 74.6 ± 3.71% | 20.8 ± 4.63% | 9.0 ± 3.53% |
| DC0051-S3 | 92.5 ± 0.47% | 59.9 ± 1.34% | 16.1 ± 2.04% | 14.6 ± 2.90% |
| DC0051-S4 | 95.2 ± 0.42% | 70.2 ± 7.01% | 18.2 ± 3.68% | 16.6 ± 4.14% |
| DC0051-S5 | 99.0 ± 0.25% | 80.0 ± 0.63% | 28.4 ± 0.74% | 20.3 ± 6.71% |
| DC0051-S6 | 95.4 ± 0.72% | 53.5 ± 14.88% | 4.0 ± 4.33% | 9.5 ± 1.64% |
| DC0051-S7 | 92.8 ± 1.92% | 50.2 ± 6.94% | 10.1 ± 5.82% | 13.4 ± 3.42% |
| DC0051-S8 | 96.7 ± 0.73% | 87.9 ± 0.78% | 56.0 ± 11.32% | 32.9 ± 2.70% |
| DC0051-S9 | 98.8 ± 0.26% | 84.1 ± 4.28% | 60.7 ± 12.57% | 13.6 ± 2.08% |

### Part B: SDS-PAGE/Western Blot Data

The disruption of Aβ 1-42, even in its monomeric form, was confirmed by a study involving the use of SDS-PAGE and Western blotting methods (not shown). In this latter study, triplicate samples of pre-fibiillized Aβ 1-42 (25 μM) was incubated at 37° C. for 3 days, alone or in the presence of the compounds or EDTA. Five micrograms of each sample was then filtered through a 0.2 μm filter. Protein recovered from the filtrate was then loaded, and ran on a 10-20% Tris-Tricine SDS-PAGE, blotted to nitrocellulose and detected using an Aβ-antibody (clone 6E10; Senetek). In this study, Aβ 1-42 was detected as a ˜4 kilodalton band (i.e. monomeric Aβ) following incubation alone, or in the presence of EDTA, at 3 days. For example, Aβ 1-42 monomers were not detected following incubation of Aβ 1-42 with compounds DC-0051, DC-0051-S1, DC-0051-S5, DC-0051-S8 and DC-0051-S9, correlating nicely with the Thioflavin T fluorometry data (described above) and suggesting that these compounds were capable of causing a disappearance of monomeric Aβ 1-42. This study confirms that these compounds are also capable of causing a disruption/removal of monomeric Aβ 1-42.

### Part C: Congo Red Binding Data

In the Congo red binding assay the ability of a test compound to alter amyloid (in this case, Aβ) binding to Congo red is quantified. In this assay, Aβ 1-42 and test compounds were incubated for 3 days and then vacuum filtered through a 0.2 μm filter. The amount of Aβ 1-42 retained in the filter was then quantified following staining of the filter with Congo red. After appropriate washing of the filter, any lowering of the Congo red color on the filter in the presence of the test compound (compared to the Congo red staining of the amyloid protein in the absence of the test compound) was indicative of the test compound's ability to diminish/alter the amount of aggregated and congophilic Aβ. This particular assay appears to be more stringent in character than Thioflavin T fluorometry, and it is more difficult to remove Congo red binding to Aβ 42 fibrils than assessed by other assays, so the % inhibitions observed even with potent compounds is usually lower than that observed as determined by other assays such as Thioflavin T fluometry.

In one study, the ability of Aβ fibrils to bind Congo red in the absence or presence of increasing amounts of the compounds or EDTA (at Aβ:test compound weight ratios of 1:0.001, 1:0.01, 1:0.1 and 1:1) was determined. The results of the 3-day incubations are presented in Table 2 below. Whereas EDTA caused no significant inhibition of Aβ 1-42 fibril binding to Congo red, the compounds (DC-0051, DC-0051-S1, S3, S4, S5, S6, S7, S8 and S9) caused a dose-dependent inhibition of Aβ binding to Congo red (Table 2). For example, compound DC-0051-S5 caused a significant 82.3+/−0.59% inhibition of Congo red binding to Aβ 1-42 fibrils when used at an Aβ:test compound wt/wt ratio of 1:1, and a 40.3+/−5.81% inhibition when used an Aβ:test compound wt/wt ratio of 1:0.1 (Table 2). Other good inhibitors as compared to the Aβ:test compound wt/wt ratio (of 1:0.1 appeared to be DC-0051-S1 (19.7+/−2.97% inhibition), DC-0051 (40.3+/−5.81% inhibition), DC-0051-S6 (17.1+/−4.94% inhibition), DC-0051-S8 (19.8+/−2.43% inhibition) and DC-0051-S9 (17.4+/−6.11% inhibition).

TABLE 2
Congo red Binding Data-Disruption of Aβ 1-42 Fibrils by Test Compounds
(% inhibition of Aβ; for Aβ:test compound at given wt/wt ratio)

| Test Compound # | 1:1 (wt/wt) | 1:0.1 (wt/wt) | 1:0.01 (wt/wt) | 1:0.001 (wt/wt) |
|---|---|---|---|---|
| EDTA (control) | 3.9 +/− 1.37% | 0.0 ± 0.76% | 0.0 +/− 0.49% | 0.0 ± 0.62% |
| DC-0051 | 76.7 +/− 1.22% | 40.3 +/− 5.81% | 3.3 +/− 0.95% | 1.7 +/− 0.10% |
| DC0051-S1 | 48.6 +/− 2.01% | 19.7 +/− 2.97% | 2.4 +/− 0.92% | 1.0 +/− 2.11% |
| DC0051-S3 | 36.2 +/− 2.51% | 16.6 +/− 1.87% | 0.0 +/− 2.11% | 0.0 +/− 2.12% |
| DC0051-S4 | 48.8 +/− 2.29% | 15.1 +/− 4.17% | 0.0 +/− 2.13% | 1.5 +/− 1.42% |
| DC0051-S5 | 82.3 +/− 0.59% | 17.5 +/− 1.23% | 0.2 +/− 1.97% | 0.0 +/− 1.37% |
| DC0051-S6 | 48.5 +/− 3.58% | 17.1 +/− 4.94% | 2.1 +/− 1.14% | 3.1 +/− 0.97% |
| DC0051-S7 | 44.6 +/− 4.59% | 8.8 +/− 1.70% | 0.0 +/− 0.29% | 2.4 +/− 2.23% |
| DC0051-S8 | 41.2 +/− 6.83% | 19.8 +/− 2.43% | 3.9 +/− 0.54% | 2.3 +/− 3.16% |
| DC0051-S9 | 60.8 +/− 2.12% | 17.4 +/− 6.11% | 3.8 +/− 3.90% | 0.0 +/− 1.27% |

### Example 7

### Additional Compounds Provided Herein are Potent Disrupters of Alzheimer's Aβ 1-42 Fibrils

The compounds prepared in the preceding Examples were found to be potent disrupters/inhibitors of Alzheimer's disease Aβ fibrils. In another set of studies, the efficacy of certain compounds provided herein (and referred to as DC-0051-B2, DC-0051-B3 and DC-0051-B4) to cause a disassembly/disruption of pre-formed amyloid fibrils of Alzheimer's disease (i.e. consisting of Aβ 1-42 fibrils) was analyzed.

### Thioflavin T Fluorometry Data

In one study, Thioflavin T fluorometry was used to determine the effects of the compounds, and of EDTA (as a negative control). In this assay Thioflavin T binds specifically to fibrillar amyloid, and this binding produces a fluorescence enhancement at 485 nm that is directly proportional to the amount of amyloid fibrils formed. The higher the fluorescence, the greater the amount of amyloid fibrils formed (Naki et al., Lab. Invest. 65:104-110, 1991; Levine III, Protein Sci. 2:404-410, 1993; Amyloid: Int. J. Exp. Clin. Invest. 2:1-6, 1995).

In this study, 25 μM of pre-fibrillized Aβ 1-42 (Bachem Inc) was incubated at 37° C. for 3 days, either alone, or in the presence of one of the compounds (DC-0051-82, DC-0051-B3 or DC-0051-B4). Following 3 days of co-incubation, 50 μl of each incubation mixture was transferred into a 96-well microtiter plate containing 150 μl of distilled water and 50 μl of a Thioflavin T solution (i.e. 500 mM Thioflavin T in 250 mM phosphate buffer, pH 6.8). The fluorescence was read at 485 nm (444 nm excitation wavelength) using an ELISA plate fluorometer after subtraction with buffer alone or compound alone, as blank).

The results of the 3-day incubations are presented below. For example, whereas EDTA caused no significant inhibition/disruption of Aβ 142 fibrils at all concentrations tested, the compounds (DC-0051-B2, DC-0051-B3, and DC-0051-B4) all caused a dose-dependent disruption/disassembly of preformed Aβ 1-42 fibrils to some extent (Table 3). The most efficacious compounds to disrupt pre-formed Aβ 1-42 fibrils as assessed by the Thioflavin T fluometry assay appeared to be DC-0051-B2. For example, compound DC-0051-B2 caused a significant (p<0.01) 65.8+/−2.01% inhibition when used at an Aβ:test compound wt/wt ratio of 1:0.1, and a significant 85.5 +/−1.27% inhibition when used at an Aβ:test compound wt/wt ratio of 1:1 (Table 3). This study indicated that the additional compounds provided herein are disrupters/inhibitors of Alzheimer's disease type Aβ fibrils, and usually exert their effects in a dose-dependent manner.

TABLE 3
Thioflavin T Fluorometry Data-Disruption of Aβ 1-42 Fibrils by Test
Compounds (% inhibition of Aβ; for Aβ:test compound at given wt/wt ratio)

| Test Compound # | 1:1 (wt/wt) | 1:0.1 (wt/wt) | 1:0.01 (wt/wt) | 1:0.001 (wt/wt) |
|---|---|---|---|---|
| EDTA (control) | 5.0 +/− 11.39% | 0.0 +/− 1.18% | 0.0 +/− 2.26% | 10.3 +/− 10.81% |
| DC-0051 | 98.5 +/− 0.56% | 88.8 +/− 0.76% | 41.1 +/− 2.52% | 18.6 +/− 8.89% |
| DC0051-B2 | 85.5 +/− 1.27% | 65.8 +/− 2.01% | 19.2 +/− 6.18% | 10.2 +/− 9.49% |
| DC0051-B3 | 17.9 +/− 16.85% | 22.2 +/− 2.63% | 1.0 +/− 1.62% | 15.7 +/− 7.34% |
| DC0051-B4 | 28.1 +/− 3.06% | 21.1 +/− 4.00% | 3.6 +/− 4.96% | 17.2 +/− 4.32% |

### Example 8

### Compounds Provided Herein are Potent Disrupters of Type 2 Diabetes IAPP Fibrils

The compounds prepared in the preceding Examples were also found to be potent disrupters/inhibitors of type 2 diabetes IAPP fibrils. In a set of studies, the efficacy of certain compounds provided herein to cause a disassembly/disruption of pre-formed amyloid fibrils of type 2 diabetes (i.e. consisting of IAPP fibrils) was analyzed.

### Part A—Thioflavin T Fluorometry Data

In one study, Thioflavin T fluorometry was used to determine the effects of the compounds, and of EDTA (as a negative control). In this assay Thioflavin T binds specifically to fibrillar amyloid, and this binding produces a fluorescence enhancement at 485 nm that is directly proportional to the amount of amyloid fibrils formed. The higher the fluorescence, the greater the amount of amyloid fibrils formed (Naki et al., Lab. Invest. 65:104-110, 1991; Levine III, Protein Sci. 2:404-410, 1993; Amyloid: Int. J. Exp. Clin. Invest. 2:1-6, 1995).

In this study, 25 μM of IAPP (Bachem Inc) was incubated at 37° C. for 3 days, either alone, or in the presence of one of the compounds or EDTA (at IAPP:test compound weight ratios of 1:1, 1:0.1, 1:0.01 or 1:0.001). Following 3 days of co-incubation, 50 μl of each incubation mixture was transferred into a 96-well microtiter plate containing 150 μl of distilled water and 50 μl of a Thioflavin T solution (i.e. 500 mM Thioflavin T in 250 mM phosphate buffer, pH 6.8). The fluorescence was read at 485 nm (444 nm excitation wavelength) using an ELISA plate fluorometer after subtraction with buffer alone or compound alone, as blank).

The results of the 3-day incubations are presented below. For example, whereas EDTA caused no significant inhibition/disruption of IAPP fibrils at all concentrations tested, the compounds (DC-0051, DC-0051-S1, S3, S4, S5, S6, S7, S8 and S9) all caused a dose-dependent disruption/disassembly of preformed IAPP fibrils to some extent (Table 4). For example, compound DC-0051-S8 caused a significant (p<0.01) 91.4+/−1.06% inhibition when used at an IAPP:test compound wt/wt ratio of 1:0.1, and a significant 52.2+/−0.45% inhibition when used at an IAPP:test compound wt/wt ratio of 1:0.01 (Table 4). Under the same conditions (i.e. IAPP:test compound wt/wt ratio of 1:0.01), compound DC-0051 caused a 63.9+/−0.56% disruption, compound DC-0051-S1 caused a 47.2+/−5.48% disruption, and compound DC-0051-S3 caused a 49.3+/−0.65% disruption. This study indicated that the compound provided herein are also potent disrupters/inhibitors of type 2 diabetes IAPP fibrils, and usually exert their effects in a dose-dependent manner.

TABLE 4
Thioflavin T Fluorometry Data-Disruption of IAPP Fibrils by Test Compounds
(% inhibition of IAPP; for IAPP:test compound at given wt/wt ratio)

| Test Compound # | 1:1 (wt/wt) | 1:0.1 (wt/wt) | 1:0.01 (wt/wt) | 1:0.001 (wt/wt) |
|---|---|---|---|---|
| EDTA (control) | 0.0 ± 1.31% | 1.6 +/− 5.86% | 4. +/− 3.152% | 0.0 +/− 0.56% |
| DC-0051 | 99.6 +/− 0.12% | 95.6 +/− 0.31% | 63.9 +/− 0.56% | 32.5 +/− 1.51% |
| DC0051-S1 | 98.5 +/− 0.12% | 86.2 +/− 1.95% | 47.2 +/− 5.48% | 6.7 +/− 0.64% |
| DC0051-S3 | 98.7 +/− 0.24% | 87.2 +/− 1.48% | 49.3 +/− 0.65% | 19.0 +/− 2.70% |
| DC0051-S4 | 97.5 +/− 0.11% | 80.2 +/− 1.59% | 36.7 +/− 0.74% | 14.3 +/− 1.57% |
| DC0051-S5 | 99.3 +/− 0.21% | 87.1 +/− 1.46% | 36.1 +/− 1.29% | 15.0 +/− 2.38% |
| DC0051-S6 | 98.7 +/− 0.52% | 74.4 +/− 12.17% | 19.7 +/− 1.64% | 0.0 +/− 1.68% |
| DC0051-S7 | 98.6 +/− 0.18% | 77.7 +/− 2.68% | 30.3 +/− 6.06% | 7.7 +/− 2.60% |
| DC0051-S8 | 99.5 +/− 0.32% | 91.4 +/− 1.06% | 52.2 +/− 0.45% | 8.8 +/− 0.55% |
| DC0051-S9 | 99.5 +/− 0.15% | 82.8 +/− 4.28% | 34.8 +/− 1.07% | 7.0 +/− 2.49% |

### Part B: Congo Red Binding Data

In the Congo red binding assay the ability of a test compound to alter amyloid (in this case, IAPP) binding to Congo red is quantified. In this assay, IAPP and test compounds were incubated for 3 days and then vacuum filtered through a 0.2 μm filter. The amount of IAPP retained in the filter was then quantified following staining of the filter with Congo red. After appropriate washing of the filter, any lowering of the Congo red color on the filter in the presence of the test compound (compared to the Congo red staining of the amyloid protein in the absence of the test compound) was indicative of the test compound's ability to diminish/alter the amount of aggregated and congophilic IAPP. This particular assay appears to be more stringent in character than Thioflavin T fluorometry, and it is more difficult to remove Congo red binding to IAPP fibrils than assessed by other assays, so the % inhibitions observed even with potent compounds is usually lower than that observed as determined by other assays such as Thioflavin T fluometry.

In one study, the ability of IAPP fibrils to bind Congo red in the absence or presence of increasing amounts of the compounds or EDTA (at IAPP:test compound weight ratios of 1:0.001, 1:0.01, 1:0.1 and 1:1) was determined. The results of the 3-day incubations are presented in Table 5 below. Whereas EDTA caused no significant inhibition of IAPP fibril binding to Congo red, the compounds (DC-0051, DC-0051-S1, S3, S4, S5, S6, S7, 88 and S9) caused a dose-dependent inhibition of IAPP binding to Congo red (Table 5). For example, compound DC-0051-S8 caused a significant 41.0+/−4.15% inhibition of Congo red binding to IAPP fibrils when used at an IAPP:test compound wt/wt ratio of 1:1, and a 26.7+/−0.82% inhibition when used an IAPP:test compound wt/wt ratio of 1:0.1 (Table 5). Other good inhibitors as compared to the IAPP:test compound wt/wt ratio of 1:0.1 appeared to be DC-0051 (51.+/−0.63% inhibition), DC-0051-S1 (24.1+/−1.99% inhibition), DC-0051-S4 (22.0+/−0.26% inhibition), and DC-0051-S9 (21.2+/−2.70% inhibition)

TABLE 5
Congo red Binding Data-Disruption of IAPP Fibrils by Test Compounds
(% inhibition of IAPP; for IAPP:test compound at given wt/wt ratio)

| | | | | 1:0.001 |
|---|---|---|---|---|
| Test Compound # | 1:1 (wt/wt) | 1:0.1 (wt/wt) | 1:0.01 (wt/wt) | (wt/wt) |
| EDTA (control) | 13.9 +/− 4.71% | 0.0 +/− 2.40% | 0.0 +/− 1.65% | 0.0 ± 1.82% |
| DC-0051 | 73.6 +/− 2.15% | 51.0 +/− 0.63% | 8.7 +/− 2.60% | 0.0 +/− 4.07% |
| DC0051-S1 | 44.1 +/− 1.03% | 24.1 +/− 1.99% | 0.0 +/− 2.55% | 0.0 +/− 3.60% |
| DC0051-S3 | 52.5 +/− 1.84% | 17.4 +/− 2.21% | 3.4 +/− 3.63% | 0.0 +/− 1.94% |
| DC0051-S4 | 30.0 +/− 1.38% | 22.0 +/− 0.26% | 2.4 +/− 3.24% | 0.0 +/− 2.89% |
| DC0051-S5 | 59.3 +/− 0.93% | 11.0 +/− 3.94% | 0.0 +/− 1.50% | 0.0 +/− 2.26% |
| DC0051-S6 | 46.0 +/− 0.65% | 7.7 +/− 5.15% | 0.0 +/− 4.57% | 0.0 +/− 1.41% |
| DC0051-S7 | 42.7 +/− 2.82% | 3.6 +/− 1.15% | 3.0 +/− 3.54% | 1.8 +/− 3.25% |
| DC0051-S8 | 41.0 +/− 4.15% | 26.7 +/− 0.82% | 0.0 +/− 5.19% | 0.3 +/− 2.37% |
| DC0051-S9 | 52.3 +/− 1.00% | 21.2 +/− 2.70% | 1.1 +/− 5.40% | 2.5 +/− 5.02% |

### Example 9

### Compounds Provided Herein are Potent Disrupters of Alpha-Synuclein Fibrils

The compounds prepared in the preceding Examples were also found to be potent disrupters/inhibitors of alpha-synuclein fibrils. In a set of studies, the efficacy of certain compounds provided herein to cause a disassembly/disruption of pre-formed amyloid-like fibrils of Parkinson's disease (i.e. consisting of alpha-synuclein fibrils) was analyzed.

### Thioflavin T Fluorometry Data

In one study, Thioflavin T fluorometry was used to determine the effects of the compounds, and of EDTA (as a negative control). In this assay Thioflavin T binds specifically to fibrillar amyloid, and this binding produces a fluorescence enhancement at 485 nm that is directly proportional to the amount of amyloid fibrils formed. The higher the fluorescence, the greater the amount of amyloid fibrils formed (Naki et al., Lab. Invest. 65:104-110, 1991; Levine III, Protein Sci. 2:404-410, 1993; Amyloid: Int. J. Exp. Clin. Invest. 2:1-6, 1995).

In this study, 25 μM of alpha-synuclein (Recombinant Peptide) was first incubated at 55° C. for 2 days with heparin (Sigma) to cause alpha-synuclein aggregation and fibril formation. Heparin, is a highly sulfated glycosaminoglycan known to cause aggregation of amyloid proteins. Following initial alpha-synuclein fibrillization, alpha-synuclein +heparin was incubated at 37° C. for 3 days, either alone, or in the presence of one of the compounds or EDTA (at α-synuclein:test compound weight ratios of 1:1, 1:0.1, 1:0.01 or 1:0.001). Following 3 days of co-incubation, 50 μl of each incubation mixture was transferred into a 96-well microtiter plate containing 150 μl of distilled water and 50 μl of a Thioflavin T solution (i.e. 500 mM Thioflavin T in 250 mM phosphate buffer, pH 6.8). The fluorescence was read at 485 nm (444 nm excitation wavelength) using an ELISA plate fluorometer after subtraction with buffer alone or compound alone, as blank).

The results of the 3-day incubations are presented below. The compounds (DC-0051, DC-0051-S1, S3, S4, S5, S6, S7, S8 and S9) all caused a dose-dependent disruption/disassembly of preformed alpha-synuclein fibrils to some extent (Table 6). For example, compound DC-0051-51 caused a significant (p<0.01) 94.5+/−2.11% inhibition when used at an α-synuclein:test compound wt/wt ratio of 1:0.1, and a significant 99.1+/−0.12% inhibition when used at an α-synuclein:test compound wt/wt ratio of 1:1 (Table 6). On the other hand, compounds DC-0051-S8 caused a significant (p<0.01) 84.6+/−0.47% inhibition when used at an α-synuclein:test compound wt/wt ratio of 1:0.1, and a significant 96.1+/−1.14% inhibition when used at an α-synuclein:test compound wt/wt ratio of 1:1 (Table 6). This study indicated that the compounds provided herein are also potent disrupters/inhibitors of Parkinson's disease alpha-synuclein fibrils, and usually exert their effects in a dose-dependent manner.

TABLE 6
Thioflavin T Fluorometry Data-Disruption of Apha-Synuclein Fibrils by Test
Compounds (% inhibition of α-synuclein; for α-synuclein:test compound at given wt/wt
ratio)

| Test Compound # | 1:1 (wt/wt) | 1:0.1 (wt/wt) | 1:0.01 (wt/wt) | 1:0.001 (wt/wt) |
|---|---|---|---|---|
| DC-0051 | 99.7 +/− 0.09% | 98.6 +/− 0.26% | 82.7 +/− 2.40% | 64.5 +/− 1.64% |
| DC0051-S1 | 99.1 +/− 0.12% | 94.5 +/− 2.11% | 55.9 +/− 13.31% | 52.9 +/− 1.34% |
| DC0051-S3 | 97.0 +/− 0.85% | 87.6 +/− 4.07% | 43.6 +/− 11.73% | 37.6 +/− 5.18% |
| DC0051-S4 | 96.0-+/− 0.50% | 86.8 +/− 1.55% | 53.7 +/− 10.98% | 41.1 +/− 6.53% |
| DC0051-S5 | 98.5 +/− 0.09% | 91.6 +/− 0.37% | 65.0 +/− 4.42% | 49.1 +/− 2.61% |
| DC0051-S6 | 96.0 +/− 1.65% | 66.6 +/− 5.77% | 46.9 +/− 5.52% | 53.3 +/− 1.70% |
| DC0051-S7 | 96.0 +/− 0.78% | 82.1 +/− 8.94% | 33.4 +/− 4.77% | 47.9 +/− 6.32% |
| DC0051-S8 | 96.1 +/− 1.14% | 84.6 +/− 0.47% | 44.1 +/− 2.19% | 38.7 +/− 4.76% |
| DC0051-S9 | 99.6 +/− 0.19% | 96.1 +/− 0.65% | 64.5 +/− 5.56% | 50.9 +/− 1.86% |

### Example 10

### Compositions of Compounds Provided Herein

The compounds provided herein, as mentioned previously, are desirably administered in the form of pharmaceutical compositions. Suitable pharmaceutical compositions, and the method of preparing them, are well-known to persons of ordinary skill in the art and are described in such treatises as Remington: The Science and Practice of Pharmacy, A. Gennaro, ed., 20th edition, Lippincott, Williams & Wilkins, Philadelphia, Pa.

Representative compositions are as follows:

### Oral Tablet Formulation

An oral tablet formulation of a compound provided herein is prepared as follows:

| | % w/w |
|---|---|
| Compound provided herein | 10.0 |
| Magnesium stearate | 0.5 |
| Starch | 2.0 |
| Hydroxypropylmethylcellulose | 1.0 |
| Microcrystalline cellulose | 86.5 |

The ingredients are mixed to homogeneity, then granulated with the aid of water, and the granulates dried. The granulate is then compressed into tablets sized to give a suitable dose of the compound. The tablet is optionally coated by applying a suspension of a film forming agent (e.g. hydroxypropylmethylcellulose), pigment (e.g. titanium dioxide), and plasticizer (e.g. diethyl phthalate), and drying the film by evaporation of the solvent. The film coat may comprise, for example, 2-6% of the tablet weight.

### Oral Capsule Formulation

The granulate from the previous section of this Example is filled into hard gelatin capsules of a size suitable to the intended dose. The capsule is banded for sealing, if desired.

### Softgel Formulation

A softgel formulation is prepared as follows:

| | % w/w |
|---|---|
| Compound provided herein | 20.0 |
| Polyethylene glycol 400 | 80.0 |

The compound is dissolved or dispersed in the polyethylene glycol, and a thickening agent added if required. A quantity of the formulation sufficient to provide the desired dose of the compound is then filled into softgels.

### Parenteral Formulation

A parenteral formulation is prepared as follows:

| | % w/w |
|---|---|
| Compound provided herein | 1.0 |
| Normal saline | 99.0 |

The compound is dissolved in the saline, and the resulting solution is sterilized and filled into vials, ampoules, and prefilled syringes, as appropriate.

### Controlled-Release Oral Formulation

A sustained release formulation may be prepared by the method of U.S. Pat. No. 4,710,384, as follows:

One Kg of a compound provided herein is coated in a modified Uni-Glatt powder coater with Dow Type 10 ethyl cellulose. The spraying solution is an 8% solution of the ethyl cellulose in 90% acetone to 10% ethanol. Castor oil is added as plasticizer in an amount equal to 20% of the ethyl cellulose present. The spraying conditions are as follows: 1) speed, 1 liter/hour; 2) flap, 10-15%; 3) inlet temperature, 50° C., 4) outlet temperature, 30° C., 5) percent of coating, 17%. The coated compound is sieved to particle sizes between 74 and 210 microns. Attention is paid to ensure a good mix of particles of different sizes within that range. Four hundred mg of the coated particles are mixed with 100 mg of starch and the mixture is compressed in a hand press to 1.5 tons to produce a 500 mg controlled release tablet.

### Example 11

### Compounds of this Invention are Potent Disrupters of Alzheimer's Aβ 1-42 Fibrils

In a set of studies, the efficacy of the compounds to cause a disassembly/disruption of pre-formed amyloid fibrils of Alzheimer's disease (i.e. consisting of Aβ 1-42 fibrils) was analyzed.

### Part A—Thioflavin T Fluorometry Data

In one study, Thioflavin T fluorometry was used to determine the effects of the compounds, and of EDTA (as a negative control). In this assay Thioflavin T binds specifically to fibrillar amyloid, and this binding produces a fluorescence enhancement at 485 nm that is directly proportional to the amount of amyloid fibrils formed. The higher the fluorescence, the greater the amount of amyloid fibrils formed (Naki et al., Lab. Invest. 65:104-110, 1991; Levine III, Protein Sci. 2:404-410, 1993; Amyloid: Int. J. Exp. Clin. Invest. 2:1-6, 1995).

In this study, 30 μg of pre-fibrillized Aβ 1-42 (rPeptide) was incubated at 37° C. for 3 days either alone, or in the presence of one of the compounds or EDTA (at Aβ:test compound weight ratios of 1:1, 1:0.1, 1:0.01 or 1:0.001). Following 3-days of co-incubation, 50 μl of each incubation mixture was transferred into a 96-well microtiter plate containing 150 μl of distilled water and 50 μl of a Thioflavin T solution (i.e. 500 mM Thioflavin T in 250 mM phosphate buffer, pH 6.8). The fluorescence was read at 485 nm (444 nm excitation wavelength) using an ELISA plate fluorometer after subtraction with buffer alone or compound alone, as blank.

The results of the 3-day incubations are presented in Table 7 below. For example, whereas EDTA caused no significant inhibition of Aβ 1-42 fibrils at all concentrations tested, the compounds all caused a dose-dependent disruption/disassembly of preformed Aβ 1-42 fibrils to some extent. The most efficacious compounds to disrupt pre-formed Aβ 1-42 fibrils appeared to be compounds DC-0051-F2 and DC-0051-F5. For example, compound F2 caused a significant (p<0.01) 95.9±0.88% inhibition when used at an Aβ:test compound wt/wt ratio of 1:0.1, and a 53.7±6.29% disruption when used at an Aβ:compound wt/wt ratio of 1:0.01. Under the same conditions (i.e. Aβ:test compound wt/wt ratio of 1:0.1), compound F5 caused a 91.8±0.22% disruption, and a 43.9±2.01% disruption when uses at an Aβ:compound wt/wt ratio of 1:0.01. This study indicated that the compounds of this invention are potent disruptors/inhibitors of Alzheimer's disease type Aβ fibrils, and usually exert their effects in a dose-dependent manner.

TABLE 7
Thioflavin T fluorometry data - disruption
of Aβ 1-42 Alzheimer's fibrils
% Inhibition Aβ (result ± S.D.) at
Aβ:test compound wt/wt ratio given

| Test | | | | |
|---|---|---|---|---|
| Compound # | 1:1 | 1:0.1 | 1:0.01 | 1:0.001 |
| EDTA | 0.0 ± 3.46 | 0.0 ± 6.02 | 2.4 ± 2.86 | 0.0 ± 1.70 |
| (control) | | | | |
| DC-0051-F1 | 98.7 ± 0.38 | 91.3 ± 1.39 | 37.1 ± 1.44 | 15.6 ± 2.01 |
| DC-0051-F2 | 99.6 ± 0.55 | 95.9 ± 0.88 | 53.7 ± 6.29 | 17.1 ± 4.32 |
| DC-0051-F3 | 99.0 ± 0.77 | 92.3 ± 0.19 | 34.6 ± 2.21 | 15.1 ± 3.81 |
| DC-0051-F4 | 99.7 ± 0.58 | 79.8 ± 0.77 | 33.4 ± 2.09 | 16.0 ± 6.64 |
| DC-0051-F5 | 99.8 ± 0.40 | 91.8 ± 0.22 | 43.9 ± 2.01 | 19.5 ± 1.49 |
| DC-0051-F6 | 99.4 ± 0.86 | 88.0 ± 1.09 | 36.8 ± 3.43 | 9.6 ± 0.96 |

### Part B: Congo Red Binding Data

In the Congo red binding assay, the ability of a test compound to alter amyloid (in this case, Aβ) binding to Congo red is quantified. In this assay, Aβ 1-42 and test compounds were incubated for 3 days and then vacuum filtered through a 0.2 μm filter. The amount of Aβ 1-42 retained in the filter was then quantitated following staining of the filter with Congo red. After appropriate washing of the filter, any lowering of the Congo red color on the filter in the presence of the test compound (compared to the Congo red staining of the amyloid protein in the absence of the test compound) was indicative of the test compound's ability to diminish/alter the amount of aggregated and congophilic Aβ.

In one study, the ability of Aβ fibrils to bind Congo red in the absence or presence of increasing amounts of the compounds or EDTA (at Aβ:test compound weight ratios of 1:1, 1:0.1, 1:0.01 or 1:0.001) was determined. The results of 3-day incubations are presented in Table 8 below. Whereas EDTA caused no significant inhibition of Aβ1-42 fibril binding to Congo red at all concentrations tested, the compounds caused a dose-dependent inhibition of Aβ binding to Congo red. For example, compound DC-0051-F2 caused a significant (p<0.01) 80.1±3.79% inhibition of Congo red binding to Aβ 1-42 fibrils when used at an Aβ:test compound wt/wt ratio of 1:1, and a significant (p<0.01) 49.9±9.13% inhibition of Congo red binding when used at an Aβ:test compound wt/wt ratio of 1:0.1. In another example, compound DC-0051-F1 caused a significant (p<0.01) 80.1±2.61% inhibition of Congo red binding to Aβ 1-42 fibrils when used at an Aβ:test compound wt/wt ratio of 1:1, and a significant (p<0.01) 27.5±2.39% inhibition of Congo red binding when used at an Aβ:test compound wt/wt ratio of 1:0.1. This study also indicated that compounds of this invention are potent inhibitors of Aβ fibril binding to Congo red, and usually exert their effects in a dose-dependent manner.

TABLE 8
Congo red binding data
% Inhibition Aβ (result ± S.D.) at Aβ:test compound wt/wt ratio given

| Test Compound # | 1:1 | 1:0.1 | 1:0.01 | 1:0.001 |
|---|---|---|---|---|
| EDTA (control) | 8.4 ± 2.74 | 0.0 ± 6.83 | 8.4 ± 4.00 | 1.2 ± 7.95 |
| DC-0051-F1 | 80.1 ± 2.61 | 27.5 ± 2.39 | 2.2 ± 7.70 | 3.7 ± 4.36 |
| DC-0051-F2 | 80.1 ± 3.79 | 49.9 ± 9.13 | 0.5 ± 5.24 | 4.9 ± 7.88 |
| DC-0051-F3 | 74.5 ± 2.69 | 23.3 ± 5.01 | 0.0 ± 3.25 | 0.0 ± 2.64 |
| DC-0051-F4 | 71.0 ± 2.39 | 10.9 ± 5.05 | 8.8 ± 5.07 | 4.3 ± 11.81 |
| DC-0051-F5 | 69.9 ± 2.06 | 25.7 ± 5.69 | 9.8 ± 2.54 | 0.0 ± 2.50 |
| DC-0051-F6 | 68.0 ± 2.11 | 18.3 ± 5.32 | 6.8 ± 1.51 | 3.7 ± 3.24 |

### Example 12

### Compounds of this Invention are Potent Disrupters of Parkinson's Disease Alpha (α) Synuclein Fibrils

The tested compounds of this invention were found also to be potent disruptors/inhibitors of Parkinson's disease α-synuclein fibrils. α-synuclein has been demonstrated to form amyloid-like fibrils when incubated at 37° C. for a few days. It is postulated to play an important role in the pathogenesis of Parkinson's disease and other synucleinopathies. In a set of studies, the efficacy of the compounds to cause a disassembly/disruption of pre-formed α-synuclein fibrils of Parkinson's disease was analyzed.

### Part A—Thioflavin T Fluorometry Data

In one study, Thioflavin T fluorometry was used to determine the effects of compounds DC-0051-F1, F2, F3, F4, F5, F6 and EDTA (as a negative control). In this assay, Thioflavin T binds specifically to α-synuclein fibrils, and this binding produces a fluorescence enhancement at 485 nm that is directly proportional to the amount of α-synuclein fibrils present. The higher the fluorescence, the greater the amount of α-synuclein fibrils present (Naki et al, Lab. Invest. 65:104-110, 1991; Levine III, Protein Sci. 2:404-410, 1993; Amyloid: Int. J. Exp. Clin. Invest. 2:1-6, 1995).

In this study, 30 μg of pre-fibrillized α-synuclein (rPeptide) was incubated at 37° C. for 3 days either alone or in the presence of compounds DC-0051-F1, F2, F3, F4, F5, F6 or EDTA (at α-synuclein:test compound weight ratios of 1:1, 1:0.1, 1:0.01 or 1:0.001). Following 3-days of co-incubation, 50 μl of each incubation mixture was transferred into a 96-well microtiter plate containing 150 μl of distilled water and 50 μl of a Thioflavin T solution (i.e. 500 mM Thioflavin T in 250 mM phosphate buffer, pH 6.8). The fluorescence was read at 485 nm (444 nm excitation wavelength) using an ELISA plate fluorometer after subtraction with buffer alone or compound alone, as blank.

The results of the 3-day incubations are presented below in Table 9. For example, whereas EDTA caused no significant inhibition of α-synuclein fibrils at all concentrations tested, compounds DC-0051-F1, F2, F3, F4, F5 and F6 all caused a dose-dependent disruption/disassembly of pre-formed α-synuclein fibrils to various extents. For example, compound F2 caused a significant (p<0.01) 94.7±2.57% inhibition when used at an α-synuclein:test compound ratio of 1:0.1, and a 61.1±1.37% disruption when used at a α-synuclein:compound wt/wt ratio of 1:0.01. Under the same conditions (i.e. α-synuclein:test compound wt/wt ratio of 1:0.1), compound F3 caused a 92.5±2.35% disruption, and a 59.6±6.39% disruption when uses at an α-synuclein:compound wt/wt ratio of 1:0.01. This study indicated that compounds of this invention are potent disruptors/inhibitors of Parkinson's disease α-synuclein fibrils, and usually exert their effects in a dose-dependent manner.

TABLE 9
Thioflavin T fluorometry data - disruption of Parkinson's disease α-synuclein
fibrils
% Inhibition α-synuclein (result ± S.D.) at α-synuclein:test compound wt/wt ratio
given

| Test Compound # | 1:1 | 1:0.1 | 1:0.01 | 1:0.001 |
|---|---|---|---|---|
| EDTA (control) | 0.0 ± 2.03 | 0.0 ± 6.97 | 0.0 ± 17.06 | 0.0 ± 4.11 |
| DC-0051-F1 | 100.0 ± 3.14 | 90.4 ± 2.07 | 58.4 ± 5.06 | 13.7 ± 6.06 |
| DC-0051-F2 | 100.0 ± 1.49 | 94.7 ± 2.57 | 61.1 ± 1.37 | 15.9 ± 7.20 |
| DC-0051-F3 | 96.8 ± 2.82 | 92.5 ± 2.35 | 59.6 ± 6.39 | 13.8 ± 5.12 |
| DC-0051-F4 | 100.0 ± 2.84 | 94.3 ± 1.22 | 40.6 ± 1.49 | 6.1 ± 9.44 |
| DC-0051-F5 | 98.9 ± 0.65 | 94.7 ± 1.70 | 59.0 ± 1.18 | 5.9 ± 10.41 |
| DC-0051-F6 | 100.0 ± 1.98 | 94.7 ± 0.68 | 56.2 ± 1.49 | 4.2 ± 5.02 |

Part B: Congo red binding data

In the Congo red binding assay, the ability of a given test compound to alter amyloid (in this case, α-synuclein) binding to Congo red is quantified. In this assay, α-synuclein and test compounds were incubated for 3 days and then vacuum filtered through a 0.2 μm filter. The amount of α-synuclein retained in the filter was then quantitated following staining of the filter with Congo red. After appropriate washing of the filter, any lowering of the Congo red color on the filter in the presence of the test compound (compared to the Congo red staining of the amyloid protein in the absence of the test compound) was indicative of the test compound's ability to diminish/alter the amount of aggregated and congophilic α-synuclein.

In one study, the ability of α-synuclein fibrils to bind Congo red in the absence or presence of increasing amounts of compounds DC-0051-F1, F2, F3, F4, F5, F6 and EDTA (at α-synuclein:test compound weight ratios of 1:1, 1:0.1, 1:0.01 or 1:0.001) was determined. The results of 3-day incubations are presented in Table 10 below. Whereas EDTA caused no significant inhibition of α-synuclein fibril binding to Congo red at all concentrations tested, the compounds caused a dose-dependent inhibition of α-synuclein binding to Congo red. For example, compound F2 caused a significant (p<0.01) 67.8±3.52% inhibition of Congo red binding to α-synuclein fibrils when used at a α-synuclein:test compound wt/wt ratio of 1:1, and a significant (p<0.01) 36.5±53.4% inhibition of Congo red binding when used at a α-synuclein:test compound wt/wt ratio of 1:0.1. In comparison, compound F1 caused a 69.8±2.67% inhibition of Congo red binding to α-synuclein fibrils when used at a α-synuclein:test compound wt/wt ratio of 1:1, and a 32.9±2.97% inhibition of Congo red binding when used at a α-synuclein:test compound wt/wt ratio of 1:0.1. This study also indicated that compounds of this invention are also potent inhibitors of Parkinson's disease type α-synuclein fibril binding to Congo red, and usually exert their effects in a dose-dependent manner.

TABLE 10
Congo red binding data - disruption of
Parkinson's disease α-synuclein fibrils
% Inhibition α-synuclein (result ± S.D.) at
α-synuclein:test compound wt/wt ratio given

| Test Compound # | 1:1 | 1:0.1 | 1:0.01 | 1:0.001 |
|---|---|---|---|---|
| EDTA (control) | 0.0 ± 5.50 | 2.2 ± 5.16 | 2.9 ± 5.0 | 3.3 ± 1.85 |
| DC-0051-F1 | 69.8 ± 2.67 | 32.9 ± 2.97 | 3.8 ± 5.82 | 0.0 ± 3.42 |
| DC-0051-F2 | 67.8 ± 3.52 | 36.5 ± 5.34 | 3.7 ± 2.83 | 0.0 ± 6.71 |
| DC-0051-F3 | 55.9 ± 2.65 | 23.3 ± 2.24 | 2.6 ± 4.90 | 0.0 ± 3.93 |
| DC-0051-F4 | 64.8 ± 5.08 | 18.0 ± 0.58 | 0.0 ± 3.44 | 6.1 ± 8.92 |
| DC-0051-F5 | 69.4 ± 0.13 | 32.7 ± 3.96 | 5.0 ± 5.47 | 0.0 ± 1.01 |
| DC-0051-F6 | 59.8 ± 3.62 | 29.9 ± 2.41 | 1.4 ± 5.69 | 0.0 ± 4.20 |

The claimed subject matter is not limited in scope by the specific embodiments described herein. Indeed, various modifications of the specific embodiments in addition to those described will become apparent to those skilled in the art from the foregoing descriptions. Such modifications are intended to fall within the scope of the appended claims. Various publications are cited herein, the disclosures of which are incorporated by reference in their entireties.

What is claimed is:

1. A compound having a formula: 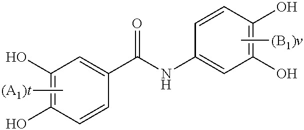
or a pharmaceutically acceptable salt thereof, where A₁ and B₁ are each independently selected from hydrogen, halogen, cyanide, cyanate, thiocyanate, selenocyanate, trifluoromethoxy, azide, nitro, ⁺NH₃, SO₃H, carboxy and haloalkyl; and t and v are each independently 1 to 3.

2. The compound of claim 1, wherein the compound has a formula selected from:

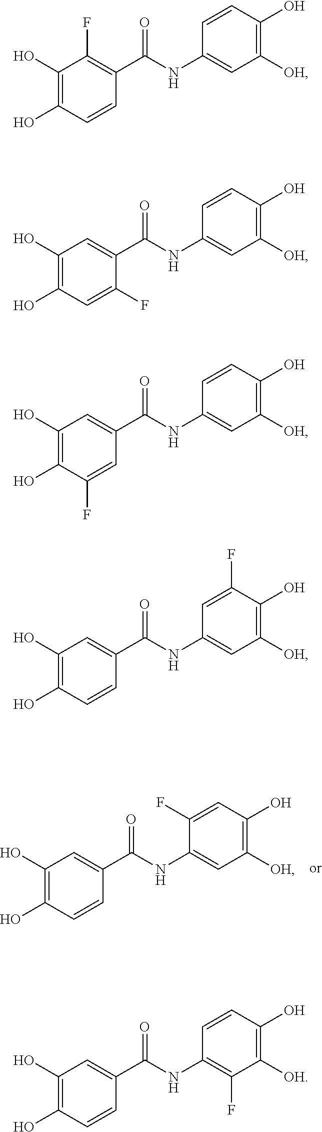

3. The compound of claim 1, wherein the compound is selected from: N-(3,4-dihydroxyphenyl)-2-fluoro-3,4-dihydroxybenzamide, N-(3,4-dihydroxyphenyl)-2-fluoro-4,5-dihydroxybenzamide, N-(3,4-dihydroxyphenyl)-3-fluoro-4,5-dihydroxybenzamide, N-(3-fluoro-4,5-dihydroxyphenyl)-3,4-dihydroxybenzamide, N-(2-fluoro-4,5-dihydroxyphenyl)-3,4-dihydroxybenzamide, and N-(2-fluoro-3,4-dihydroxyphenyl)-3,4-dihydroxybenzamide.

4. The compound of claim 1, wherein A₁ and B₁ are each independently selected from F, Cl, Br, I, cyanide, cyanate, thiocyanate, selenocyanate, trifluoromethoxy, and azide.

5. The compound of claim 1 where A₁ or B₁ is radioisotopically labeled.

6. The compound of claim 1 where A₁ or B₁ is ¹⁸F.

7. A pharmaceutical composition comprising the compound of claim 1 or a pharmaceutically acceptable salt thereof, and a pharmaceutically acceptable excipient.

8. A method of treating the formation, deposition, accumulation, or persistence of amyloid protein, comprising administering an effective amount of the compound of claim 1.

9. The method of claim 8, where the amyloid protein is Aβ amyloid fibrils.

10. The method of claim 8, where the amyloid protein is IAPP amyloid fibrils.

11. The method of claim 8, where the compound administered is in an amount between 0.1 mg/Kg/day and 1000 mg/Kg/day.

12. The method of claim 8, where the compound is administered in an amount between 1 mg/Kg/day and 100 mg/Kg/day.

13. The method of claim 8, where amount of compound administered is in an amount between 10 mg/Kg/day and 100 mg/Kg/day.

14. A method of treating the formation, deposition, accumulation, or persistence of α-synuclein protein, comprising administering an effective amount of the compound of claim 1.

15. The method of claim 14, where the compound administered is in an amount between 0.1 mg/Kg/day and 1000 mg/Kg/day.

16. The method of claim 14, where the compound is administered in an amount between 1 mg/Kg/day and 100 mg/Kg/day.

17. The method of claim 14, where amount of compound administered is in an amount between 10 mg/Kg/day and 100 mg/Kg/day.

18. An amyloid imaging agent comprising the compound of claim 1.

19. A method of in vivo imaging of amyloid deposits, comprising introducing into a mammal a detectable quantity of the compound of claim 1 which has been radiolabeled; allowing sufficient time for the labeled compound to become associated with amyloid deposits and detecting the labeled compound associated with one or more amyloid deposits.

20. The method of claim 19 wherein the step of detecting the labeled compound is by positron emission tomography or single photon emission computed tomography.

21. An α-synuclein imaging agent comprising the compound of claim 1.

22. A method of in vivo imaging of α-synuclein deposits, comprising introducing into a mammal a detectable quantity of the compound of claim 1 which has been radiolabeled; allowing sufficient time for the labeled compound to become associated with α-synuclein deposits; and detecting the labeled compound associated with one or more α-synuclein deposits.

23. The method of claim 22 wherein the step of detecting the labeled compound is by positron emission tomography or single photon emission computed tomography.
